(19) United States
(12) Patent Application Publication
(10) Pub. No.: US 2009/0221644 A1
(43) Pub. Date: Sep. 3, 2009

(54) Gpcr Agonists

(76) Inventors: Stuart Edward Bradley, Oxford (GB); Matthew Colin Thor Fyfe, Oxford (GB); Lisa Sarah Bertram, Oxford (GB); William Gattrell, Oxford (GB); Martin James Procter, Oxford (GB); Chrystelle Marie Rasamison, Oxford (GB); Colin Peter Sambrook-Smith, Oxford (GB); Vilasben Kanji Shah, Oxford (GB); David French Stonehouse, Oxford (GB); Simon Andrew Swain, Oxford (GB); Gerard Hugh Thomas, Oxford (GB)

Correspondence Address: OSI PHARMACEUTICALS, INC., 41 PINELAWN ROAD, MELVILLE, NY, 11747, US

(21) Appl. No.: 11/922,830

(22) PCT Filed: Jun. 29, 2006
(86) PCT No.: PCT/GB2006/050177

§ 371 Date: Mar. 20, 2009

(30) Foreign Application Priority Data
Jun. 30, 2005 (GB) 0513258.4
Jun. 30, 2005 (GB) 0513272.5
Mar. 20, 2006 (GB) 0605540.4

(51) Int. Cl. A61K 31/443; C07D 405/12; A61P 3/10

(52) U.S. Cl. 514/337; 546/284.1

(57) ABSTRACT

Compounds of formula (I): or pharmaceutically acceptable salts thereof, are GPCR agonists and are useful as for the treatment of obesity and diabetes.

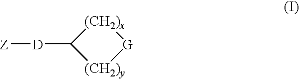

### BACKGROUND OF THE INVENTION

The present invention is directed to G-protein coupled receptor (GPCR) agonists. In particular, the present invention is directed to GPCR agonists that are useful for the treatment of obesity, e.g. as regulators of satiety, and for the treatment of diabetes.

Obesity is characterized by an excessive adipose tissue mass relative to body size. Clinically, body fat mass is estimated by the body mass index (BMI; weight(kg)/height(m)²), or waist circumference. Individuals are considered obese when the BMI is greater than 30 and there are established medical consequences of being overweight. It has been an accepted medical view for some time that an increased body weight, especially as a result of abdominal body fat, is associated with an increased risk for diabetes, hypertension, heart disease, and numerous other health complications, such as arthritis, stroke, gallbladder disease, muscular and respiratory problems, back pain and even certain cancers.

Pharmacological approaches to the treatment of obesity have been mainly concerned with reducing fat mass by altering the balance between energy intake and expenditure. Many studies have clearly established the link between adiposity and the brain circuitry involved in the regulation of energy homeostasis. Direct and indirect evidence suggest that serotonergic, dopaminergic, adrenergic, cholinergic, endocannabinoid, opioid, and histaminergic pathways in addition to many neuropeptide pathways (e.g. neuropeptide Y and melanocortins) are implicated in the central control of energy intake and expenditure. Hypothalamic centres are also able to sense peripheral hormones involved in the maintenance of body weight and degree of adiposity, such as insulin and leptin, and fat tissue derived peptides.

Drugs aimed at the pathophysiology associated with insulin dependent Type I diabetes and non-insulin dependent Type II diabetes have many potential side effects and do not adequately address the dyslipidaemia and hyperglycaemia in a high proportion of patients. Treatment is often focused at individual patient needs using diet, exercise, hypoglycaemic agents and insulin, but there is a continuing need for novel antidiabetic agents, particularly ones that may be better tolerated with fewer adverse effects.

Similarly, metabolic syndrome (syndrome X) which is characterized by hypertension and its associated pathologies including atherosclerosis, lipidemia, hyperlipidemia and hypercholesterolemia have been associated with decreased insulin sensitivity which can lead to abnormal blood sugar levels when challenged. Myocardial ischemia and microvascular disease is an established morbidity associated with untreated or poorly controlled metabolic syndrome.

There is a continuing need for novel antiobesity and antidiabetic agents, particularly ones that are well tolerated with few adverse effects.

GPR119 (previously referred to as GPR116) is a GPCR identified as SNORF25 in WO00/50562 which discloses both the human and rat receptors, U.S. Pat. No. 6,468,756 also discloses the mouse receptor (accession numbers: AAN95194 (human), AAN95195 (rat) and ANN95196 (mouse)).

In humans, GPR119 is expressed in the pancreas, small intestine, colon and adipose tissue. The expression profile of the human GPR119 receptor indicates its potential utility as a target for the treatment of obesity and diabetes.

International patent application WO2005/061489 (published after the priority date of the present application) discloses heterocyclic derivatives as GPR119 receptor agonists.

The present invention relates to agonists of GPR119 which are useful for the treatment of obesity e.g. as peripheral regulators of satiety, and for the treatment of diabetes.

### SUMMARY OF THE INVENTION

Compounds of formula (I):

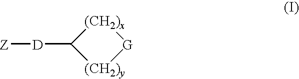

or pharmaceutically acceptable salts thereof, are agonists of GPR119 and are useful for the prophylactic or therapeutic treatment of obesity and diabetes.

### DETAILED DESCRIPTION OF THE INVENTION

The present invention is directed to a compound of formula (I):

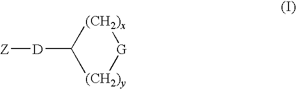

or a pharmaceutically acceptable salt or N-oxide thereof, wherein:

Z represents a group:

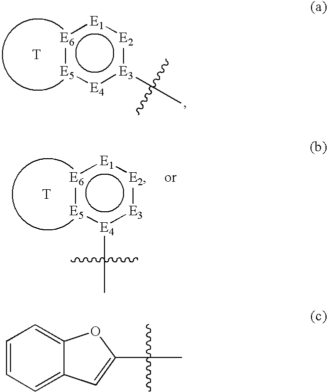

wherein E₁ to E₆ may independently represent either C/CH or N; T represents a five or six membered aryl or nitrogen containing heteroaryl ring, with the proviso that when Z represents the group (a), T does not represent:

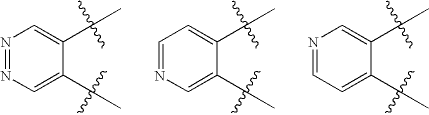

the group Z may optionally be substituted by one or more groups (e.g. one, two or three groups) selected from halogen, CF₃, C₁₋₄alkoxy, NR⁴R⁴⁴, S(O)ₘR⁴, SO₂NR⁴R⁴⁴, CONR⁴R⁴⁴, NR¹⁰CONR⁴R⁴⁴, NR⁴⁴COR⁴, NR¹⁰SO₂R⁴, nitro, cyano, or a 5- or 6-membered heteroaryl ring; or C₁₋₄alkyl, C alkenyl, or C₂₋₄alkynyl, which may be optionally substituted by hydroxy, NR⁴R⁴⁴, oxo or C₁₋₄alkoxy;

m is 0, 1 or 2;

D represents a group -B-Q-A-, wherein:

Q is a 5- or 6-membered heteroaromatic ring;

A is (CH₂)ₙ, where one CH₂ group may be replaced by O, S, C(O), CH(OH) CH(halo) CH(NR²R³), S(O), S(O)₂ or NR³; two CH₂ groups may be replaced by CH═CH, C(O)O, C(O)S, SC(O), C(O)NR² or OC(O); or three CH₂ groups may be replaced by C(O)CH₂S, C(O)CH₂C(OH) or C(O)CH₂C(O);

n is 0, 1, 2, 3, 4, 5, or 6;

B is a bond, —CH₂═CH₂— or (CH₂)ⱼ;

j is 1, 2 or 3;

or D represents -M-U—V—, wherein:

M and V are independently a bond, an unbranched or a branched C₁₋₃ alkylene or an unbranched or a branched C₂₋₃ alkenylene;

U is selected from CH₂, O, S, CH(OH), CH(halo), CH═CH, C(O), C(O)O, C(O)S, SC(O), C(O)CH₂S, C(O)CH₂C(OH), C(O)CH₂C(O), OC(O), NR², CH(NR²R²²), C(O)NR², S(O) and S(O)₂;

G is CHR⁸ or NR¹;

R¹ is C(O)OR⁵, C(O)NR⁵R¹⁰, C(O)NR⁵R⁵⁵, C₁₋₄alkylene-C(O)OR⁵, C(O)C(O)OR⁵, S(O)₂R⁵, C(O)R⁵ or P(O)(O-Ph)₂; or heterocyclyl or heteroaryl, either of which may optionally be substituted by one or two groups selected from C₁₋₄alkyl, C₁₋₄alkoxy or halogen;

R², R²² and R³ are independently hydrogen or C₁₋₄alkyl;

R⁴ and R⁴⁴ are independently hydrogen, C₁₋₄alkyl, C₃₋₇cycloalkyl, or aryl, which may optionally be substituted with 1 or 2 substituents selected from halo, C₁₋₄alkyl, CF₃, hydroxy, C₁₋₄alkoxy, cyano, and S(O)₂Me; or, taken together, R⁴ and R⁴⁴ may form a 5- or 6-membered heterocyclic ring;

R⁵ and R⁵⁵ are independently C₁₋₈alkyl, C₂₋₈alkenyl or C₂₋₈alkynyl, any of which may be optionally substituted by one or more halo atoms, NR⁶R⁶⁶, OR⁶, C(O)OR⁶, OC(O)R⁶ or cyano, and may contain a CH₂ group that is replaced by O or S; or a C₃ Cycloalkyl, aryl, heterocyclyl, heteroaryl, C₁₋₄alkyleneC₃₋₇cycloalkyl, C₁₋₄alkylenearyl, C₁₋₄alkyleneheterocyclyl or C₁₋₄ alkyleneheteroaryl, any of which may be substituted with one or more substituents selected from halo, C₁₋₄alkyl, C₁₋₄fluoroalkyl, OR⁷, CN, NR⁷R⁷⁷, SO₂Me, NO₂ or C(O)OR⁷;

R⁶, R⁶⁶, R⁷, and R⁷⁷ each independently are hydrogen or C₁₋₄allyl; or, taken together, R⁶ and R⁶⁶ or R⁷ and R⁷⁷ may independently form a 5- or 6-membered heterocyclic ring;

R⁸ is C₃₋₆alkyl;

R¹⁰ is hydrogen or C₁₋₄alkyl;

x is 0, 1, 2 or 3; and

y is 1, 2, 3, 4 or 5; provided that x+y is 2, 3, 4 or 5.

The molecular weight of the compounds of formula (I) is preferably less than 800, more preferably less than 600, even more preferably less than 500.

In one embodiment of the invention D represents -B-Q-A-. In a second embodiment of the invention D represents -M-U—V—.

When D represents -B-Q-A-:

B preferably represents a bond.

n preferably represents 0, 1 or 2, more preferably 1 or 2.

Exemplary A groups include —CH₂—O—. A is preferably CH₂, CH₂O or CH₂NR³.

Q is preferably a heteroaromatic ring containing up to 3 heteroatoms selected from N, O and S.

Q is preferably a 5-membered heteroaromatic ring containing up to three heteroatoms selected from O, N and S of the formula:

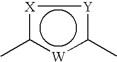

wherein W, X and Y represent the positions of the heteroatom(s) or otherwise represent CH.

Particular heteroaromatic rings which Q may represent include oxadiazole, oxazole, isoxazole, thiadiazole, thiazole and pyrazole.

Preferably two of W, X and Y are N, and the other is O.

W is preferably N.

The heteroaromatic ring described by Q is preferably oxadiazolyl, more preferably [1,2,4]oxadiazolyl.

n is preferably 0, 1 or 2, especially 1 or 2.

When D represents -M-U—V—:

-M-U—V— preferably represents a 2 to 5 atom chain.

U is preferably CH₂, O or NR², more preferably O.

M and V are preferably independently C₁₋₃ alkylene.

In one embodiment of the invention Z represents the group (a). In a second embodiment of the invention Z represents the group (b). In a third embodiment of the invention Z represents the group (c).

Suitably, the group Z may optionally be substituted by one or more groups selected from: halogen, CF₃, C₁₋₄alkoxy, NR⁴R⁴⁴, SC₁₋₄alkyl, S(O)C₁₋₄alkyl, SO₂C₁₋₄alkyl, SO₂NR⁴R⁴⁴, CONR⁴R⁴⁴, NR¹⁰CONR⁴R⁴⁴, NR¹⁰COR⁴, NR¹⁰SO₂R⁴, nitro, cyano, or a 5- or 6-membered heteroaryl ring; or C₁₋₄alkyl, C₂₋₄alkenyl, or C₂₋₄alkynyl, which may be optionally substituted by hydroxyl, NR⁴R⁴⁴, oxo or C₁₋₄alkoxy. In particular Z may optionally be substituted by one or more groups selected from: halo, CF₃, C₁₋₄alkoxy, NR⁴R⁴⁴, SC₁₋₄alkyl, S(O)C₁₋₄alkyl, S(O)₂C₁₋₄alkyl or cyano; or C₁₋₄alkyl, C₂₋₄alkenyl, or C₂alkynyl, which may be optionally substituted by hydroxy, NR⁴R⁴⁴, oxo or C₁₋₄alkoxy.

When the group Z is substituted by S(O)ₘR⁴, suitably the substituent group is S(O)ₘC₁₋₄alkyl.

Suitably, R¹ is C(O)OR⁵, C(O)NR⁵R¹⁰, C₁₋₄alkylene-C(O)OR⁵, C(O)C(O)OR⁵, S(O)₂R⁵, C(O)R⁵ or P(O)(O-Ph)₂; or heterocyclyl or heteroaryl, either of which may optionally be substituted by one or two groups selected from C₁₋₄alkyl, C₁₋₄alkoxy or halogen.

Suitably, when R⁵ represents C₃₋₅cycloalkyl it is optionally substituted by C₁₋₄alkyl.

G is preferably NR¹.

R¹ is preferably C(O)OR⁵, C(O)NR⁵R¹⁰, C₁₋₄alkylene-C(O)OR⁵, C(O)C(O)OR⁵, heterocyclyl, heteroaryl, S(O)₂R⁵, C(O)R⁵ or P(O)(O-Ph)₂; especially C(O)OR⁵, C(O)NR⁵R¹⁰, C₁₋₄alkyl-C(O)OR⁵, heteroaryl, S(O)₂R⁵ or C(O)R⁵; in particular C(O)OR⁵, C(O)NR⁵R¹⁰, heteroaryl, S(O)₂R⁵ or C(O)R⁵. More preferably, R¹ is C(O)OR⁵, C(O)NR⁵R¹⁰ or heteroaryl. R¹ is most preferably C(O)OR⁵. When R¹ is heteroaryl the heteroaryl ring is preferably a 5- or 6-membered heteroaryl ring, for example pyrimidinyl or pyridyl, especially pyrimidinyl e.g. pyrimidin-2-yl.

Suitably R⁴ and R⁴⁴ are independently hydrogen, C₁₋₄alkyl, C₃₋₇cycloalkyl, or aryl, which may optionally be substituted with 1 or 2 substituents selected from halo, C₁₋₄alkyl, CF₃, C₁₋₄alkoxy, cyano, and S(O)₂Me; or, taken together, R⁴ and R⁴⁴ may form a 5- or 6-membered heterocyclic ring.

Preferably R⁵ represents C₁₋₈alkyl, C₂₋₈alkenyl or C₂₋₈alkynyl optionally substituted by one or more halo atoms or cyano, and may contain a CH₂ group that is replaced by O or S; or a C₃₋₇cycloalkyl, aryl or C₁₋₄alkylC₃₋₇cycloalkyl, any of which may be substituted with one or more substituents selected from halo, C₁₋₄alkyl, C₁₋₄fluoroalkyl, OR⁷, CN, NR⁷R⁷⁷, NO₂ or C(O)OC₁₋₄alkyl. More preferably R⁵ represents C₁₋₈alkyl, C₂₋₈alkenyl or C₂₋₈alkynyl optionally substituted by one or more halo atoms or cyano, and may contain a CH₂ group that is replaced by O or S; or a C₃₋₇cycloalkyl or aryl, either of which may be substituted with one or more substituents selected from halo, C₁₋₄alkyl, C₁₋₄fluoroalkyl, OR⁷, CN, NR⁷R⁷⁷, NO₂ or C(O)OC₁₋₄alkyl. Most preferred R⁵ groups are C₂₋₅alkyl (optionally substituted by one or more halo atoms or cyano, and may contain a CH₂ group that is replaced by O or S), such as C₃₋₅alkyl (optionally substituted by one or more halo atoms or cyano, and which may contain a CH₂ group that is replaced by O or S); or C₃₋₅cycloalkyl (optionally substituted by halo, C₁₋₄alkyl, C₁₋₄fluoroalkyl, OR⁷, CN, NR⁷R⁷⁷, NO₂ or C(O)OC₁₋₄alkyl). In one embodiment of the invention the group represented by R⁵ is unsubstituted.

Suitably R⁵⁵ represents hydrogen or C₁₋₄alkyl.

In one embodiment of the invention x+y is 2, 3, or 4. In a preferred embodiment of the invention x and y each represent 1. In a more preferred embodiment of the invention x and y each represent 2.

It is to be understood that the bicyclic ring system defined by the Z groups (a) and (b) can represent heteroaromatic ring systems containing a ring-junction nitrogen, such as indolizine and imidazo[1,2-a]pyridine, i.e. where one of E₅ and E₆ is N. In addition, it is to be understood that when E₁ to E₆ represent either C/CH the position in the ring will determine if the group is C or CH. Thus, when bound directly to D or when fused to the adjoining ring, E^{#} represents C. Alternatively, if E^{#} is not connected directly to D or fused to the adjoining ring, then it represents CH. In addition, in any CH group the H may be optionally replaced by one of the substituents recited for the group Z. Preferably the optionally substituted bicyclic ring represented by Z groups (a) and (b) will contain 0 to 4 nitrogen ring atoms. Exemplary bicyclic rings containing zero nitrogen atoms include naphthalene. Exemplary bicylic rings containing one nitrogen ring atom include indole, isoindole, indolizine, quinoline and isoquinoline. Exemplary bicyclic rings containing two nitrogen atoms include indazole, benzimidazole, imidazo[1,2-a]pyridine and pyrrolo[1,2-c]pyrimidine. Exemplary bicyclic rings containing three nitrogen atoms include benzotriazole. Exemplary bicyclic rings containing four nitrogen atoms include purine.

Examples of optional substituents for the group Z include one or more groups, e.g. one, two or three groups, selected from: halogen, CF₃, C₁₋₄alkoxy e.g. methoxy, NR⁴R⁴⁴, SC₁₋₄alkyl e.g. methylsulfanyl, S(O)C₁₋₄alkyl e.g. methanesulfinyl, SO₂C₁₋₄alkyl e.g. methanesulfonyl, SO₂NR⁴R⁴⁴, CONR⁴R⁴⁴, NR¹⁰CONR⁴R⁴⁴, NR¹⁰COR⁴, NR¹⁰SO₂R⁴, nitro, cyano, or a 5- or 6 membered heteroaryl ring, or C₁₋₄alkyl, C₂₋₄alkenyl, or C₂₋₄alkynyl, which may be optionally substituted by hydroxyl, NR⁴R⁴⁴, oxo or C₁₋₄alkoxy.

Where it is stated that a particular function may optionally be substituted by one or more other groups, suitably the number of substituent groups will be one, two or three, e.g. one or two.

While the preferred groups for each variable have generally been listed above separately for each variable, preferred compounds of this invention include those in which several or each variable in formula (I) is selected from the preferred, more preferred or particularly listed groups for each variable. Therefore, this invention is intended to include all combinations of preferred, more preferred and particularly listed groups.

Specific compounds of the invention which may be mentioned are those included in the Examples and pharmaceutically acceptable salts thereof.

Specific compounds of the invention which may be mentioned are 4-[2-(5-methanesulfonylbenzofuran-2-yl)ethyl]piperidine-1-carboxylic acid tert-butyl ester and 4-[2-(5-methanesulfinylbenzofuran-2-yl)ethyl]piperidine-1-carboxylic acid tert-butyl ester.

The following provisos may optionally be used (individually or in any combination) to exclude certain compounds from the scope of formula (I):

(i) when x and y both represent 2, the group -M-U—V— does not represent —C—C(O)— or —C(O)—.

(ii) when x represents 0, y represents 4, G represents NC(O)R⁵ and R⁵ represents substituted or unsubstituted phenyl, 5-membered heterocyclyl, 6-membered heterocyclyl, bicyclic aryl or bicyclic heteroaryl, the group -M-U—V— does not represent —CH₂—.

(iii) when G represents NR¹, -M-U—V— does not represent:

—C(O)—O—R—;

—O—R—;

—C(O)—NH—R—; or

—NH—R;

where R represents C₁₋₆alkyl, C₃₋₇cycloalkyl, C₁₋₆alkylC₃₋₇cycloalkyl or C₃₋₇cycloalkyl C₁₋₆alkyl.

(iv) when Z represents the group (c), it is not substituted at the 7-position by C₁₋₄alkoxy or SC₁₋₄alkyl:

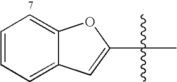

(v) when Z represents the group (c), G represents N—C(O)—O-tButyl, x represents 2 and y represents 2, -M-U—V— does not represent —C(O)—.

(vi) when x represents 0, y represents 3 and G represents N—C(O)—R⁵, the group Z does not represent (c) substituted by a cyano at either the 5 or the 6 position.

(vii) when -M-U—V— represents —C(O)NH— and Z represents the group (c), Z is not substituted by a cyano group at the 5 position.

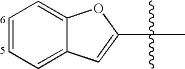

As used herein, unless stated otherwise, “alkyl” as well as other groups having the prefix “alk” such as, for example, alkenyl, alkynyl, and the like, means carbon chains which may be linear or branched or combinations thereof. Examples of alkyl groups include methyl, ethyl, propyl, isopropyl, butyl, sec- and tert-butyl, pentyl, hexyl, heptyl and the like. “Alkenyl”, “alkynyl” and other like terms include carbon chains having at least one unsaturated carbon-carbon bond.

The term “fluoroalkyl” includes alkyl groups substituted by one or more fluorine atoms, e.g. CH₂F, CHF₂ and CF₃.

The term “cycloalkyl” means carbocycles containing no heteroatoms, and includes monocyclic and bicyclic saturated and partially saturated carbocycles. Examples of cycloalkyl include cyclopropyl, cyclobutyl, cyclopentyl, cyclohexyl and cycloheptyl. Examples of partially saturated cycloalkyl groups include cyclohexene and indane. Cycloalkyl groups will typically contain 3 to 10 ring carbon atoms in total, e.g. 3 to 6, or 8 to 10.

The term “halo” includes fluorine, chlorine, bromine, and iodine atoms (in particular fluorine or chlorine).

The term “aryl” includes phenyl and naphthyl, in particular phenyl.

Unless otherwise indicated the term “heterocyclyl” and “heterocyclic ring” includes 4- to 10-membered monocyclic and bicyclic saturated rings, e.g. 4- to 7-membered monocyclic saturated rings, containing up to three heteroatoms selected from N, O and S. Examples of heterocyclic rings include oxetane, tetrahydrofuran, tetrahydropyran, oxepane, oxocane, thietane, tetrahydrothiophene, tetrahydrothiopyran, thiepane, thiocane, azetidine, pyrrolidine, piperidine, azepane, azocane, [1,3]dioxane, oxazolidine, piperazine, and the like. Other examples of heterocyclic rings include the oxidised forms of the sulfur-containing rings. Thus, tetrahydrothiophene 1-oxide, tetrahydrothiophene 1,1-dioxide, tetrahydrothiopyran 1-oxide, and tetrahydrothiopyran 1,1-dioxide are also considered to be heterocyclic rings.

Unless otherwise stated, the term “heteroaryl” includes mono- and bicyclic 5- to 10-membered, e.g. monocyclic 5- or 6-membered, heteroaryl rings containing up to 4 heteroatoms selected from N, O and S. Examples of such heteroaryl rings are furyl, thienyl, pyrrolyl, pyrazolyl, imidazolyl, oxazolyl, isoxazolyl, thiazolyl, isothiazolyl, triazolyl, oxadiazolyl, thiadiazolyl, tetrazolyl, pyridinyl, pyridazinyl, pyrimidinyl, pyrazinyl and triazinyl. Bicyclic heteroaryl groups include bicyclic heteroaromatic groups where a 5- or 6-membered heteroaryl ring is fused to a phenyl or another heteroaromatic group. Examples of such bicyclic heteroaromatic rings are benzofuran, benzothiophene, indole, benzoxazole, benzothiazole, indazole, benzimidazole, benzotriazole, quinoline, isoquinoline, quinazoline, quinoxaline and purine. Preferred heteroaryl groups are monocyclic 5- or 6-membered, heteroaryl rings containing up to 4 heteroatoms selected from N, O and S.

Compounds described herein may contain one or more asymmetric centers and may thus give rise to diastereomers and optical isomers. The present invention includes all such possible diastereomers as well as their racemic mixtures, their substantially pure resolved enantiomers, all possible geometric isomers, and pharmaceutically acceptable salts thereof. The above formula (I) is shown without a definitive stereochemistry at certain positions. The present invention includes all stereoisomers of formula (I) and pharmaceutically acceptable salts thereof. Further, mixtures of stereoisomers as well as isolated specific stereoisomers are also included. During the course of the synthetic procedures used to prepare such compounds, or in using racemization or epimerization procedures known to those skilled in the art, the products of such procedures can be a mixture of stereoisomers.

When a tautomer of the compound of formula (I) exists, the present invention includes any possible tautomers and pharmaceutically acceptable salts thereof, and mixtures thereof, except where specifically drawn or stated otherwise.

When the compound of formula (I) and pharmaceutically acceptable salts thereof exist in the form of solvates or polymorphic forms, the present invention includes any possible solvates and polymorphic forms. A type of a solvent that forms the solvate is not particularly limited so long as the solvent is pharmacologically acceptable. For example, water, ethanol, propanol, acetone or the like can be used.

The term “pharmaceutically acceptable salts” refers to salts prepared from pharmaceutically acceptable non-toxic bases or acids. When the compound of the present invention is acidic, its corresponding salt can be conveniently prepared from pharmaceutically acceptable non-toxic bases, including inorganic bases and organic bases. Salts derived from such inorganic bases include aluminum, ammonium, calcium, copper (ic and ous), ferric, ferrous, lithium, magnesium, potassium, sodium, zinc and the like salts. Particularly preferred are the ammonium, calcium, magnesium, potassium and sodium salts. Salts derived from pharmaceutically acceptable organic non-toxic bases include salts of primary, secondary, and tertiary amines, as well as cyclic amines and substituted amines such as naturally occurring and synthesized substituted amines. Other pharmaceutically acceptable organic non-toxic bases from which salts can be formed include arginine, betaine, caffeine, choline, N′,N′-dibenzylethylenediamine, diethylamine, 2-diethylaminoethanol, 2-dimethylaminoethanol, ethanolamine, ethylenediamine, N-ethylmorpholine, N-ethylpiperidine, glucamine, glucosamine, histidine, hydrabamine, isopropylamine, lysine, methylglucamine, morpholine, piperazine, piperidine, polyamine resins, procaine, purines, theobromine, triethylamine, trimethylamine, tripropylamine, tromethamine and the like.

When the compound of the present invention is basic, its corresponding salt can be conveniently prepared from pharmaceutically acceptable non-toxic acids, including inorganic and organic acids. Such acids include, for example, acetic, benzenesulfonic, benzoic, camphorsulfonic, citric, ethanesulfonic, fumaric, gluconic, glutamic, hydrobromic, hydrochloric, isethionic, lactic, maleic, malic, mandelic, methanesulfonic, mucic, nitric, pamoic, pantothenic, phosphoric, succinic, sulfuric, tartaric, p-toluenesulfonic acid and the like.

Since the compounds of formula (I) are intended for pharmaceutical use they are preferably provided in substantially pure form, for example at least 60% pure, more suitably at least 75% pure, especially at least 98% pure (% are on a weight for weight basis).

Compounds of formula (I) in which U is CO₂, COS, or CONR² can be prepared by condensing the appropriate acid (I) with an alcohol, thiol, or amine (III), as shown in Scheme 1 where E is O, S, or NR², using a typical reagent for such a condensation reaction, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The acids (II) and alcohols, thiols, and amines (III) are either commercially available or are prepared easily using known techniques.

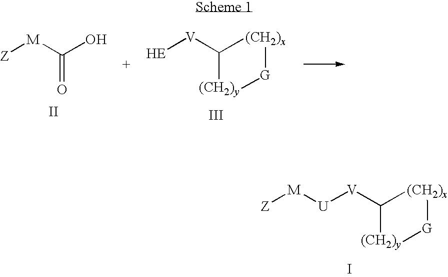

Compounds of formula (I) in which U is SCO or OCO can be prepared by condensing the appropriate thiol or alcohol (IV) with the appropriate acid (V), as shown in Scheme 2 where E is S or O, employing a reagent typically used for effecting such reactions, e.g., EDCI (Pottorf, R. S.; Szeto, P. In Handbook of Reagents for Organic Synthesis: Activating Agents and Protecting Groups; Pearson, A. J., Roush, W. R., Eds.; Wiley: Chichester, 1999; pp 186-188). The alcohols and thiols (IV), as well as acids (V), are either commercially available or are prepared easily using known techniques.

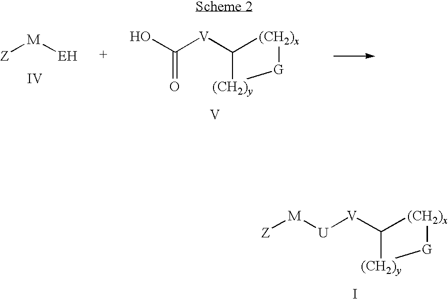

Compounds of formula (I) in which U is S, NR² or O can be prepared by alkylating the appropriate thiol, amine or alcohol (IV) with the appropriate alkyl halide or sulfonate ester (VI), as shown in Scheme 3 where E is S or O and LG is chloro, bromo, iodo, alkanesulfonate, or arenesulfonate. The reaction is typically carried out using a base, e.g., potassium tert-butoxide (Hall, S. E., et al. J. Med. Chem. 1989, 32, 974-984). The alcohols, amines and thiols (IV), as well as the alkyl halides or sulfonates (VI), are either commercially available or are made easily using known techniques. The compounds of formula (I) where U is SO or SO₂ can easily be obtained from the compounds of formula (I) where U is S by oxidation with, for example, mCPBA (Fyfe, M. C. T. et al. International Patent Publication WO 04/72031).

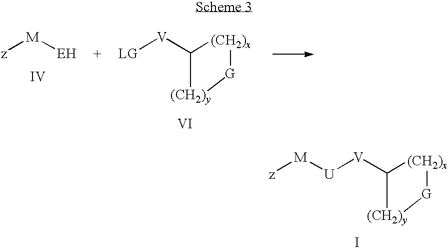

Compounds of formula (I) in which M is C₂₋₃ alkenylene can be prepared by a Wittig reaction between the appropriate phosphonium salt (VII) and the appropriate aldehyde (VIII), as indicated in Scheme 4 where m is 1 or 2 and n is 0 or 1 with the proviso that m+n<3. As an alternative to the approach described in Scheme 4, the compounds of formula (I) in which M is C₂₋₃ alkenylene can be prepared by a Wittig reaction between the appropriate aldehyde (IX) and the appropriate phosphonium salt (X), as indicated in Scheme 5 where q is 0 or 1 and r is 1 or 2 with the proviso that q+r<3. The reactions are carried out in the presence of a suitable base, e.g., NaOMe or LiHMDS (March, J. Advanced Organic Chemistry, 4th edn.; Wiley: New York, 1992; pp 956-963). The phosphonium salts (VII) and (X), as well as the aldehydes (VIII) and (IX), are either commercially available or are made easily using known techniques. The compounds of formula (I) where M is C₂₋₃ alkylene can easily be synthesized from the compounds of formula (I) where M is C₂₋₃ alkenylene by a hydrogenation reaction using, for example, palladium on charcoal as a catalyst.

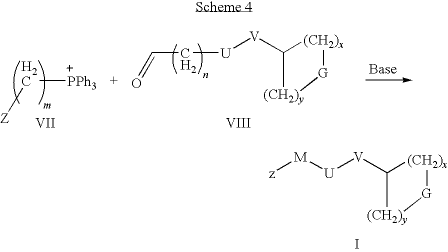

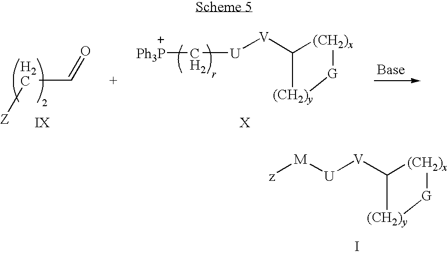

Compounds of the formula (I) where M is a bond and U is S, NR² or O can be prepared by condensation of the appropriate heteroaryl halide (XI), where Z is an electron-deficient heteroaryl moiety, with the appropriate alcohol, amine or thiol (III), as depicted in Scheme 6 where Hal represents a halogen and E is S, NR² or O. The reaction is carried out in the presence of a suitable basic system, e.g., potassium hydroxide and potassium carbonate in the presence of tris(3,6-dioxaheptyl)amine (Ballesteros, P.; Claramunt, R. M.; Elguero, J. Tetrahedron 1987, 43, 2557-2564). The heteroaryl halides (XI) and alcohols/amines/thiols (III) are either commercially available or are made easily using known techniques.

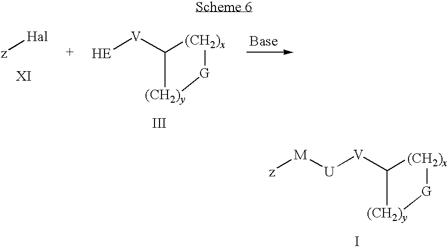

Compounds of the formula (I) where G is NC(O)OR⁵, NC(O)NR⁵R⁵⁵, NC(O)R⁵, or NC(O)C(O)OR⁵ can be prepared by the route shown in Scheme 7, where an amine of formula (XII) is condensed with an acyl chloride of formula (XIII) where A is O, NR⁵, a bond, or C(O)O. The reaction is carried out in the presence of a suitable base, such as triethylamine (Picard, F., et al. J. Med. Chem. 2002, 45, 3406-3417). Compounds of the formula (I) where G is NCONR⁴R⁵ and R⁵ is hydrogen may also be prepared by reacting the amine (XII) with a suitable isocyanate O═C═N—R⁵ (Boswell, R. F., Jr., et al. J. Med. Chem. 1974, 17, 1000-1008). Compounds of the formula (I) where G is N—C₁₋₄alkylene-C(O)OR⁵ may be prepared by alkylating the amine (XII) with the appropriate α-haloester (Rooney, C. S. et al. J. Med. Chem. 1983, 26, 700-714). The amine (XII) is generally derived from its N-tert-butoxycarbonyl precursor—prepared by one of the routes outlined in Schemes 1-6—by deprotection with an acid, e.g., trifluoroacetic acid (Fyfe, M. C. T. et al. International Patent Publication WO 04/72031).

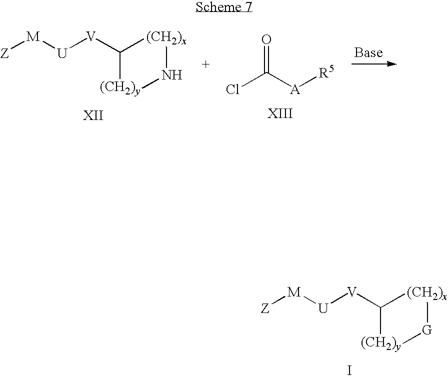

Compounds of the formula (I) where G is N-heteroaryl may be prepared by condensation of amine (XII) with a heteroaryl chloride of formula (XIV), as illustrated in Scheme 8 (Barillari, C. et al. Eur. J. Org. Chem. 2001, 4737-4741; Birch, A. M. et al. J. Med. Chem. 1999, 42, 3342-3355).

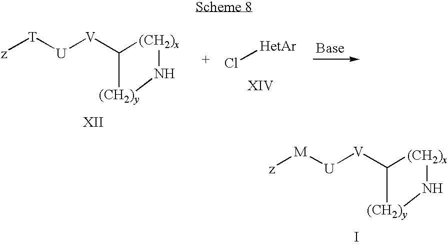

Compounds of the formula (I) where Z is substituted by CN can be prepared from the corresponding unsubstituted bicylic ring by the Reissert reaction (Fife, W. K. J. Org. Chem. 1983, 48, 1375-1377). Similar reactions can be used to prepare the compounds where Z is substituted by a halogen (Walters, M. A.; Shay, J. J. Tetrahedron Lett. 1995, 36, 7575-7578). The compounds where Z is substituted by halogen can be transformed into the corresponding compounds where Z is substituted by Clot alkyl by transition metal-catalysed cross-coupling reactions (Fürstner, A., et al. J. Am. Chem. Soc. 2002, 124, 13856-13863).

Other compounds of formula (I) can be prepared as described below, in which, for illustrative purposes, -Q is shown as a group of the formula:

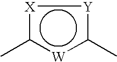

and Z, B, A, G, x, y, W, X and Y are as defined above.

The compounds of formula (I), in which X=N, Y=0 and W=N, may be prepared according to the method illustrated in Scheme 9. The nitrites of formula 2 are either commercially available or can be synthesised using known techniques. Compounds of formula 2 are treated with hydroxylamine in a suitable solvent, such as ethanol-water, at elevated temperature, to afford amidoximes of formula 3 (synthesis of amidoximes is further described by A. R Martin et al., J. Med. Chem., 2001, 44, 1560). Compounds of formula 3 are subsequently condensed with acids of formula 4, which are themselves either commercially available or can be readily synthesised using known techniques. The condensation firstly entails activation of compounds of formula 4 by, for example, formation of the mixed anhydride, in which the acid is treated with a chloroformate, such as isobutylchloroformate, in the presence of a suitable base, such as triethylamine, in a suitable solvent, such as THF or toluene, followed by addition of compounds of formula 3. Alternatively, compounds of formula 4 may be activated by conversion to the acid halide, generated by treatment of the acid with, for example, oxalyl chloride in a suitable solvent, such as CH₂Cl₂-DMF. The intermediates arising from the condensation of amidoximes of formula 3 and acids of formula 4 are dissolved in an appropriate solvent, such as toluene or xylene, and heated under reflux, with concomitant removal of water by Dean-Stark apparatus or by molecular sieves, to form oxadiazoles of formula (I). Alternatively, amidoximes of formula 3 can firstly be treated with a suitable base, for example sodium hydride, in an appropriate solvent, such as THF, and subsequently esters of formula 5. Heating of this mixture also generates oxadiazoles of formula (I) (this process is further illustrated by R. H. Mach et al, Bioorg. Med. Chem., 2001, 9, 3113).

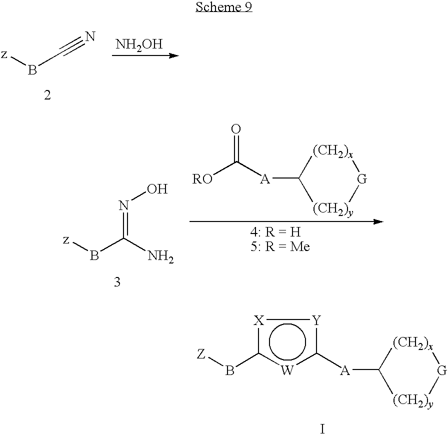

Compounds of formula (I) in which X=O, Y=N and W=N may be prepared according to the method outlined in Scheme 10. The nitriles of formula 6 are either commercially available or can be synthesised using known techniques. These are converted to the corresponding amidoximes of formula 7, as described above, and subsequently condensed with acids of formula 8, which are commercially available or can readily be synthesised by those skilled in the art. This condensation is performed in a fashion analogous to that described in Scheme 1, to afford the corresponding oxadiazoles of formula (I).

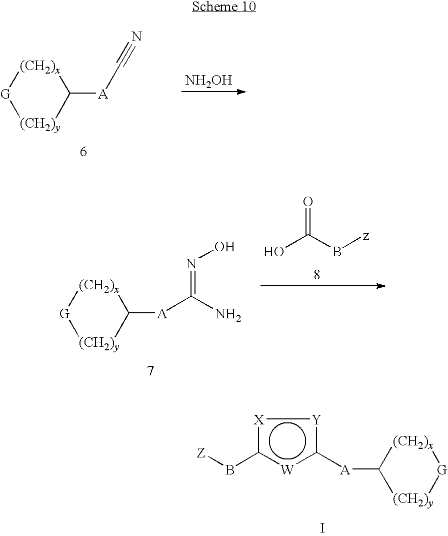

Compounds of formula (I) in which X=N, Y=N and W=O can be synthesised as outlined in Scheme 11. The acyl chlorides of formula 9 are either commercially available or may be synthesised using known methods. The acid hydrazides of formula 10 can be readily obtained by, for example, treating an ethanolic solution of the corresponding ester with hydrazine (for further details see K. M. Kahn et al, Bioorg. Med. Chem., 2003, 11, 1381). Treating the acyl chlorides of formula 9 with the acid hydrazides of formula 10 in a suitable solvent, such as pyridine, affords compounds of formula 11 (further illustrated by V. N. Kerr et al, J. Am. Chem. Soc., 1960, 82, 186), which are then converted by POCl₃ at elevated temperature to compounds of formula (I) (this process is further described by S-A. Chen et al, J. Am. Chem. Soc., 2001, 123, 2296). Similarly, compounds of formula (I) where X=Y=W=N can be prepared via the condensation of the amidrazone analogue of 10 with the appropriate activated carboxylic acid derivative, such as 9. The reactive groups in this reaction may be exchanged, i.e., an amidrazone of formula Z-B—C(═NH)NHNH₂ can form a compound of formula (I) by condensation with an activated carboxylic acid derivative LG-C(═O-A-cycle where LG is halogen or oxycarbonyl (P. H. Olesen et al., J. Med. Chem., 2003, 46, 3333-3341).

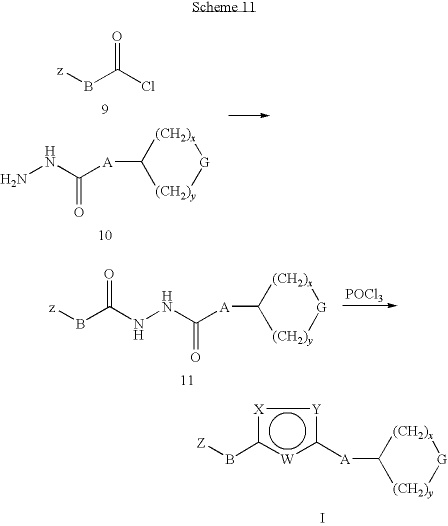

Compounds of formula (I) where X=N, Y=N, and W=S can also be prepared from compounds of formula 11 by heating with Lawesson's reagent in a suitable solvent, such as toluene or acetonitrile (D. Alker et al., J. Med. Chem., 1989, 32, 2381-2388). Compounds of formula (I) where X=S, Y=N and W=N can be formed from compounds of formula 12 (Scheme 12) which are commercially available, or can be readily synthesised from the corresponding carbonyl compound and Lawesson's reagent under standard conditions. Treating a compound of formula 12 with a compound of formula 13 in a suitable solvent such as dichloromethane at about 20° C. gives compounds of formula 14. Compounds of formula 13 can be obtained by treating the corresponding dimethylamide with Meerwein's reagent (for details see M. Brown U.S. Pat. No. 3,092,637). Compounds of formula 14 are then cyclised using hydroxylamine-O-sulfonic acid in the presence of a base, such as pyridine, in a suitable solvent such as methanol (for further details, see A. MacLeod et al, J. Med. Chem., 1990, 33, 2052).

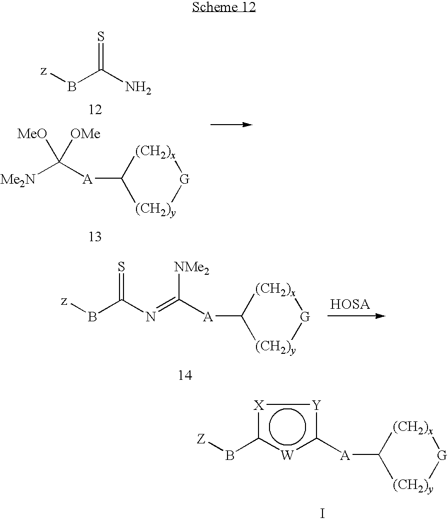

The regioisomeric derivatives of formula (I), where X=N, Y=S and W=N, can be formed in a similar manner by reversing the functionality of the reactants so the Z fragment contains the acetal moiety and the G containing cycle fragment contains the thiocarbonyl.

Compounds of formula (I) where W=O, X=N and Y=CH can be formed from compounds of formula 15 (Scheme 13). Compounds of formula 15 are commercially available or synthesised using known techniques. Chlorides of formula 16 are commercially available, or can readily be formed by chlorinating the corresponding ketone using standard conditions, for example, bubbling chlorine gas through a methanol solution of the ketone (for further details see R. Gallucci & R. Going, J. Org. Chem., 1981, 46, 2532). Mixing a compound of formula 15 with a chloride of formula 16 in a suitable solvent, such as toluene, with heating, for instance at about 100° C. gives compounds of formula (I) (for further information, see A. Hassner et al, Tetrahedron, 1989, 45, 6249). Compounds of formula (I) where W=O, X=CH and Y=N can be formed is a similar fashion by reversing the functionality of the reactants so the Z fragment contains the haloketone moiety and the G containing cycle fragment contains the C(O)NH₂.

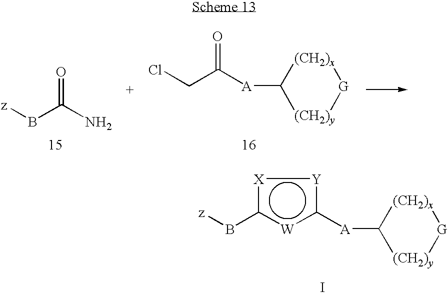

Alternatively, compounds of formula (I) where X=S, W=N and Y=CH can also be formed from compounds of formula 16. Heating an compound of formula 15 with phosphorus pentasulfide, followed by the addition of a compound of formula 16 followed by further heating gives compounds of formula (I) (for further details, see R Kurkjy & E. Brown, J. Am. Chem. Soc., 1952, 74, 5778). The regioisomeric compounds where X=CH, W=N and Y=S can be formed is a similar fashion by reversing the functionality of the reactants, so the R¹ fragment contains the haloketone moiety and the G containing cycle fragment contains the C(O)NH₂.

Compounds of formula I where W=N, X=O and Y=CH can be formed from compounds of formula 15 and formula 17 (Scheme 14) under similar conditions to those outlined for Scheme 5. Compounds of formula I where W=S, X=N and Y=CH can also be formed from compounds of formula 15 and formula 17 using the conditions involving phosphorus pentasulfide described above.

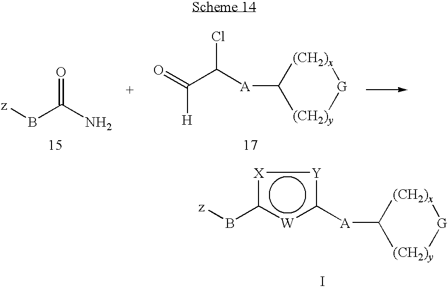

Compounds of formula (I) where X=O, Y=N and W=CH, and where X=N, Y=O and W=CH and can be formed from compounds of formula 20 (Scheme 15). Acylation of compounds of formula 18 with a compound of formula 19, where V is alkoxide or chloride, can occur under standard conditions, for example, deprotonation of ketone 18 with a suitable base, such as lithium diisopropylamide or potassium ethoxide, in a suitable solvent, such as tetrahydrofuran, generally at low temperature. Treatment of compounds of formula 20 with hydroxylamine, in a suitable solvent, such as ethanol, at elevated temperature, for example 75° C., yields compounds of formula (I) as a mixture of both regioisomers of the isoxazole. Using standard separation techniques, such as chromatography on silica gel, the individual isomers can be isolated (for further details, see M. Rowley et al, J. Med. Chem., 1997, 40, 2374).

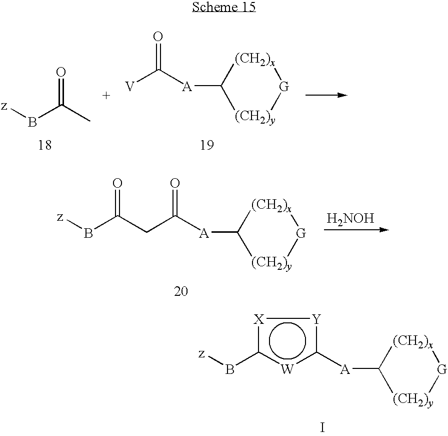

Compounds of formula (I) where X=S, Y=N and W=CH can be formed by hydrogenation of a compound of formula (I) where X=O, Y=N and W=CH, with platinum oxide in a suitable solvent such as ethanol, followed by heating with phosphorus pentasulfide to give compounds of formula (I) where X=S, Y=N and W=CH (for further details, see G. Wiegand et al, J. Med. Chem., 1971, 14, 1015). For details of the synthesis of the regioisomer where X=N, Y=S and W=CH also see G. Wiegand ibid.

Compounds of formula (I) where X=N, Y=N and W=CH can be formed from compounds of formula 20. Treatment of compounds of formula 20 with hydrazine in a suitable solvent, such as methanol, would give rise to compounds of formula (I) where X=N, Y=N and W=CH (this process is further illustrated by R. Baker et al., J. Med. Chem., 1997, 40, 2374).

Compounds of formula (I) in which X=CH, Y=N and W=N can be synthesised as described in Scheme 16. Bromides of formula 23 are either commercially available or may be synthesised from the corresponding ketone by, for example, treating an aqueous solution of the ketone with Br₂ and HBr (as described by J. Y. Becker et al, Tetrahedron Lett., 2001, 42, 1571). The amidines of formula 22 may be synthesised by known methods, for example by treatment of the corresponding alkyl imidates of formula 21 with ammonia in a suitable solvent, such as ethanol (as detailed by D. A. Pearson et al, J. Med. Chem., 1996, 39, 1372). The imidates of formula 21 may in turn be generated by, for example, treatment of the corresponding nitrile with HCl in a suitable solvent, such as methanol (for further details see J. P. Lokensgard et al, J. Org. Chem., 1985, 50, 5609). Reaction of amidines of formula 22 with bromides of formula 23 in a suitable solvent, such as DMF, affords compounds of formula (I) (illustrated by N. J. Liverton et al, J. Med. Chem., 1999, 42, 2180).

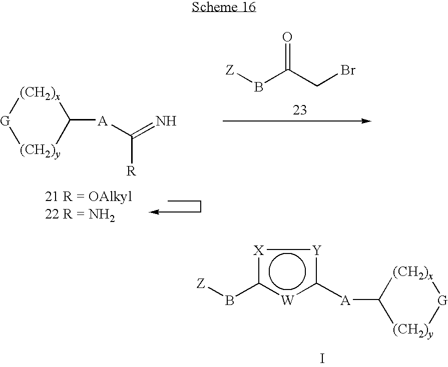

The regioisomeric compounds where X=N, Y=CH and W=N can be formed in a similar fashion by reversing the functionality of the reactants, so the Z fragment contains the amidine moiety and the G fragment contains the bromide.

Compounds of formula (I) in which X=CH, Y=CH and W=N can be synthesised as illustrated in Scheme 17. Diketones of formula 25 are readily accessible by, for example, the condensation of ketones of formula 24, which are commercially available or are readily synthesised using known techniques, with bromides of formula 23 in a suitable solvent, such as benzene using an appropriate catalyst. Illustrative examples are described by O. G. Kulinkovich et al, Synthesis, 2000, 9, 1259. Using a Paal-Knorr reaction, diketones of formula 25 may be treated with, for example, ammonium carbonate in a suitable solvent, such as ethanol at elevated temperature (for further details see R. A. Jones et al, Tetrahedron, 1996, 52, 8707) to afford compounds of formula (I).

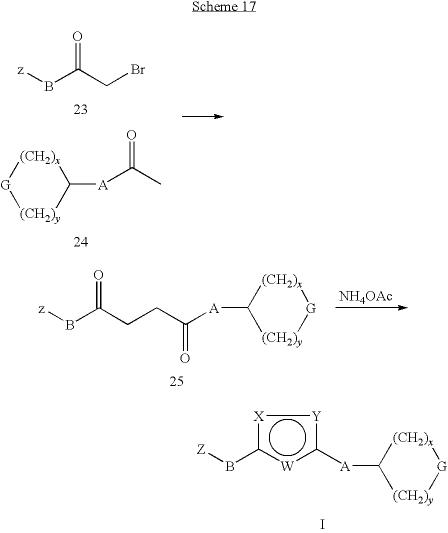

Compounds of formula (I) in which G contains either a carbamate or a sulfonamide group may be synthesised as described in Scheme 18. Compounds of formula 26, in which P represents a suitable protecting group, for example tert-butoxycarbonyl (Boc), may be synthesised as outlined in Schemes 1-9 above. The protecting group is firstly removed under suitable conditions to afford compounds of formula 27. In the case of the Boc group this can be achieved by treatment of compounds of formula 26 with a suitable acid, such as trifluoroacetic acid, in an appropriate solvent, such as CH₂Cl₂. Treatment of compounds of formula 27 with chloroformates of formula 28, which are generally commercially available or can be readily synthesised, in a suitable solvent, such as CH₂Cl₂, in the presence of a suitable base, such as triethylamine, affords compounds of formula (I). Similarly, compounds of formula 27 may be reacted with sulfonyl chlorides of formula 29, which are generally commercially available or can readily be synthesised, in a suitable solvent, such as CH₂Cl₂, in the presence of a suitable base, such as triethylamine, to afford compounds of formula (I). Compounds of formula (I) in which G contains a urea moiety may be prepared by reacting a compound of formula 27 with an isocyanate of formula O═C—N—R³. Furthermore, compounds of formula (I) in which G a heteroaryl group may be prepared by reacting the amine 27 with the appropriate heteroaryl chloride or bromide under Pd(0) catalysis in the presence of a suitable ligand and base (Urgaonkar, S.; Hu, J.-H.; Verkade, J. G. J. Org. Chem. 2003, 68, 8416-8423).

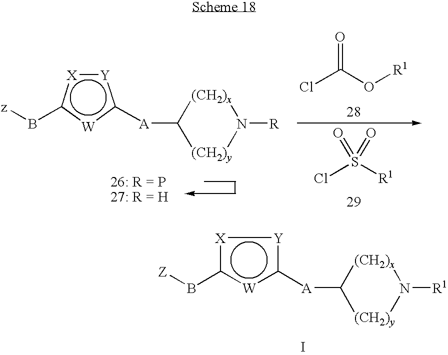

Compounds of formula (I) in which G contains an amide group may be synthesised from compounds of formula 27 and a suitable acid (R³COOH), or activated derivative thereof, in an amide bond forming reaction.

Compounds of formula (I) where A contains a NR⁵ group where R⁵ is hydrogen can be further transformed into compounds of formula (I) where R⁵ is C₁₋₄alkyl group using standard techniques known to those with skill in the art.

Compounds of the formula (I) where Z is optionally substituted with CN can be prepared from the corresponding unsubstituted Z by the Reissert reaction (Fife, W. K. J. Org. Chem. 1983, 48, 1375-1377). Similar reactions can be used to prepare the compounds where Z is optionally substituted with halogen (Walters, M. A.; Shay, J. J. Tetrahedron Lett. 1995, 36, 7575-7578). The compounds where Z is optionally substituted with halogen can be transformed into the corresponding compounds where Z optionally substituted with C₁₋₄alkyl by transition metal-catalysed cross-coupling reactions (Fürstner, A., et al. J. Am. Chem. Soc. 2002, 124, 13856-13863).

Compounds of formula (I) and where X=N, Y=N, U=N and W=CH can be synthesised as shown in Scheme 19 below. Illustrative examples are described by M. Meldal et al Journal of Organic Chemistry (2002), 67(9), 3057-3064. Azides of formula 30 are either commercially available or may be synthesised, for example, from the displacement of the corresponding halides with azide ion using known techniques; or synthesised from the corresponding amine derivative via reaction with sodium nitrite in acidic media. The alkynes of formula 31 may be commercial or synthesised by known methods, for example by reaction of acetylide ions with boranes (see Journal of Organic Chemistry (1981), 46(11) 2311-2314) or aldehydes or ketones.

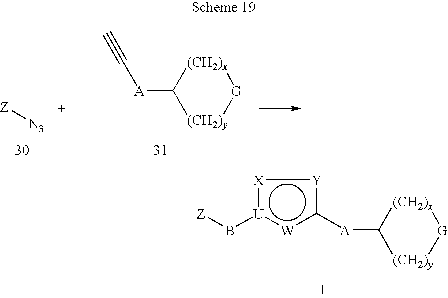

Compounds of formula (I) and where X=N, Y=CH, U=N and W=CH can be synthesised as shown in Scheme 20 by reaction of 1,3-dicarbonyl compounds of formula 33 (or their equivalents, such as enol ethers) with hydrazines of formula 32. The hydrazines of formula 32 may be commercial or synthesised by known methods, for example by reaction of the corresponding amine with sodium nitrite and reacting the resulting diazonium salt with a reducing agent such as sodium sulfite.

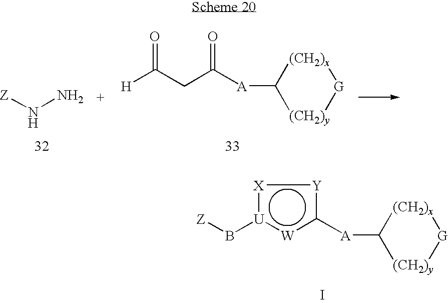

Other compounds of formula (I) may be prepared by methods analogous to those described above or by methods known per se.

Further details for the preparation of the compounds of formula (I) are found in the examples.

The compounds of formula (I) may be prepared singly or as compound libraries comprising at least 2, for example 5 to 1,000, compounds and more preferably 10 to 100 compounds of formula (I). Compound libraries may be prepared by a combinatorial “split and mix” approach or by multiple parallel synthesis using either solution or solid phase chemistry, using procedures known to those skilled in the art.

During the synthesis of the compounds of formula (I), labile functional groups in the intermediate compounds, e.g. hydroxy, carboxy and amino groups, may be protected. The protecting groups may be removed at any stage in the synthesis of the compounds of formula (I) or may be present on the final compound of formula (I). A comprehensive discussion of the ways in which various labile functional groups may be protected and methods for cleaving the resulting protected derivatives is given in, for example, Protective Groups in Organic Chemistry, T. W. Greene and P. G. M. Wuts, (1991) Wiley-Interscience, New York, 2^{nd} edition. Suitably, protecting groups will be removed from the compounds of formula (I).

Any novel intermediates, such as those defined above, may be of use in the synthesis of compounds of formula (I) and are therefore also included within the scope of the invention, for example compounds of formula (XVI) and salts and protected derivatives thereof:

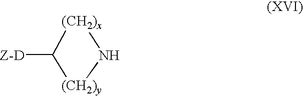

wherein Z, D, x and y are as defined for formula (I) with the proviso that when Z represents the group (c), x represents 2 and y represents 2, -M-U—V— does not represent —CH₂—NH—CH₂— or —C(O)NH—.

Exemplary groups of compounds of formula p(V′) include:

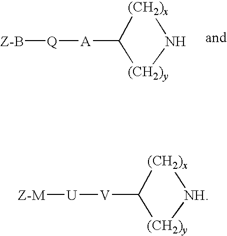

wherein the variable groups are as defined above for compounds of formula (I).

As indicated above the compounds of formula (I) are useful as GPR119 agonists, e.g. for the treatment and/or prophylaxis of obesity and diabetes. For such use the compounds of formula (I) will generally be administered in the form of a pharmaceutical composition.

The invention also provides a compound of formula (I), or a pharmaceutically acceptable salt thereof, for use as a pharmaceutical.

The invention also provides a pharmaceutical composition comprising a compound of formula (I), in combination with a pharmaceutically acceptable carrier.

Preferably the composition is comprised of a pharmaceutically acceptable carrier and a non-toxic therapeutically effective amount of a compound of formula (I), or a pharmaceutically acceptable salt thereof.

Moreover, the invention also provides a pharmaceutical composition for the treatment of disease by modulating GPR119, resulting in the prophylactic or therapeutic treatment of obesity, e.g. by regulating satiety, or for the treatment of diabetes, comprising a pharmaceutically acceptable carrier and a non-toxic therapeutically effective amount of compound of formula (I), or a pharmaceutically acceptable salt thereof.

The pharmaceutical compositions may optionally comprise other therapeutic ingredients or adjuvants. The compositions include compositions suitable for oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions may be conveniently presented in unit dosage form and prepared by any of the methods well known in the art of pharmacy.

In practice, the compounds of formula (I), or pharmaceutically acceptable salts thereof, can be combined as the active ingredient in intimate admixture with a pharmaceutical carrier according to conventional pharmaceutical compounding techniques. The carrier may take a wide variety of forms depending on the form of preparation desired for administration, e.g. oral or parenteral (including intravenous).

Thus, the pharmaceutical compositions can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion, or as a water-in-oil liquid emulsion. In addition to the common dosage forms set out above, the compound of formula (I), or a pharmaceutically acceptable salt thereof, may also be administered by controlled release means and/or delivery devices. The compositions may be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The compounds of formula (I), or pharmaceutically acceptable salts thereof, can also be included in pharmaceutical compositions in combination with one or more other therapeutically active compounds.

The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen.

In preparing the compositions for oral dosage form, any convenient pharmaceutical media may be employed. For example, water, glycols, oils, alcohols, flavoring agents, preservatives, coloring agents, and the like may be used to form oral liquid preparations such as suspensions, elixirs and solutions; while carriers such as starches, sugars, microcrystalline cellulose, diluents, granulating agents, lubricants, binders, disintegrating agents, and the like may be used to form oral solid preparations such as powders, capsules and tablets. Because of their ease of administration, tablets and capsules are the preferred oral dosage units whereby solid pharmaceutical carriers are employed. Optionally, tablets may be coated by standard aqueous or nonaqueous techniques.

A tablet containing the composition of this invention may be prepared by compression or molding, optionally with one or more accessory ingredients or adjuvants. Compressed tablets may be prepared by compressing, in a suitable machine, the active ingredient in a free-flowing form such as powder or granules, optionally mixed with a binder, lubricant, inert diluent, surface active or dispersing agent. Molded tablets may be made by molding in a suitable machine, a mixture of the powdered compound moistened with an inert liquid diluent. Each tablet preferably contains from about 0.05 mg to about 5 g of the active ingredient and each cachet or capsule preferably containing from about 0.05 mg to about 5 g of the active ingredient.

For example, a formulation intended for the oral administration to humans may contain from about 0.5 mg to about 5 g of active agent, compounded with an appropriate and convenient amount of carrier material which may vary from about 5 to about 95 percent of the total composition. Unit dosage forms will generally contain between from about 1 mg to about 2 g of the active ingredient, typically 25 mg, 50 mg, 100 mg, 200 mg, 300 mg, 400 mg, 500 mg, 600 mg, 800 mg, or 1000 mg.

Pharmaceutical compositions of the present invention suitable for parenteral administration may be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. In all cases, the final injectable form must be sterile and must be effectively fluid for easy syringability. The pharmaceutical compositions must be stable under the conditions of manufacture and storage; thus, preferably should be preserved against the contaminating action of microorganisms such as bacteria and fungi. The carrier can be a solvent or dispersion medium containing, for example, water, ethanol, polyol (e.g. glycerol, propylene glycol and liquid polyethylene glycol), vegetable oils, and suitable mixtures thereof.

Pharmaceutical compositions of the present invention can be in a form suitable for topical use such as, for example, an aerosol, cream, ointment, lotion, dusting powder, or the like. Further, the compositions can be in a form suitable for use in transdermal devices. These formulations may be prepared, using a compound of formula (I), or a pharmaceutically acceptable salt thereof, via conventional processing methods. As an example, a cream or ointment is prepared by admixing hydrophilic material and water, together with about 5 wt % to about 10 wt % of the compound, to produce a cream or ointment having a desired consistency.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories may be conveniently formed by first admixing the composition with the softened or melted carrier(s) followed by chilling and shaping in molds.

In addition to the aforementioned carrier ingredients, the pharmaceutical formulations described above may include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a compound of formula (I), or pharmaceutically acceptable salts thereof, may also be prepared in powder or liquid concentrate form.

Generally, dosage levels on the order of 0.01 mg/kg to about 150 mg/kg of body weight per day are useful in the treatment of the above-indicated conditions, or alternatively about 0.5 mg to about 7 g per patient per day. For example, obesity may be effectively treated by the administration of from about 0.01 to 50 mg of the compound per kilogram of body weight per day, or alternatively about 0.5 mg to about 3.5 g per patient per day.

It is understood, however, that the specific dose level for any particular patient will depend upon a variety of factors including the age, body weight, general health, sex, diet, time of administration, route of administration, rate of excretion, drug combination and the severity of the particular disease undergoing therapy.

The compounds of formula (I) may be used in the treatment of diseases or conditions in which GPR119 plays a role.

Thus the invention also provides a method for the treatment of a disease or condition in which GPR119 plays a role comprising a step of administering to a subject in need thereof an effective amount of a compound of formula (I), or a pharmaceutically acceptable salt thereof. Diseases or conditions in which GPR119 plays a role include obesity and diabetes. In the context of the present application the treatment of obesity is intended to encompass the treatment of diseases or conditions such as obesity and other eating disorders associated with excessive food intake e.g. by reduction of appetite and body weight, maintenance of weight reduction and prevention of rebound and diabetes (including Type 1 and Type 2 diabetes, impaired glucose tolerance, insulin resistance and diabetic complications such as neuropathy, nephropathy, retinopathy, cataracts, cardiovascular complications and dyslipidaemia). And the treatment of patients who have an abnormal sensitivity to ingested fats leading to functional dyspepsia. The compounds of the invention may also be used for treating metabolic diseases such as metabolic syndrome (syndrome X), impaired glucose tolerance, hyperlipidemia, hypertriglyceridemia, hypercholesterolemia, low HDL levels and hypertension.

The compounds of the invention may offer advantages over compounds acting via different mechanisms for the treatment of the above mentioned disorders in that they may offer beta-cell protection, increased cAMP and insulin secretion and also slow gastric emptying.

The invention also provides a method for the regulation of satiety comprising a step of administering to a subject in need thereof an effective amount of a compound of formula (I), or a pharmaceutically acceptable salt thereof.

The invention also provides a method for the treatment of obesity comprising a step of administering to a subject in need thereof an effective amount of a compound of formula (I), or a pharmaceutically acceptable salt thereof.

The invention also provides a method for the treatment of diabetes, including Type 1 and Type 2 diabetes, particularly type 2 diabetes, comprising a step of administering to a patient in need thereof an effective amount of a compound of formula (I), or a pharmaceutically acceptable salt thereof.

The invention also provides a method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound of formula (I), or a pharmaceutically acceptable salt thereof.

The invention also provides a compound of formula (I), or a pharmaceutically acceptable salt thereof, for use in the treatment of a condition as defined above.

The invention also provides the use of a compound of formula (I), or a pharmaceutically acceptable salt thereof, in the manufacture of a medicament for the treatment of a condition as defined above.

In the methods of the invention the term “treatment” includes both therapeutic and prophylactic treatment.

The compounds of formula (I), or pharmaceutically acceptable salts thereof, may be administered alone or in combination with one or more other therapeutically active compounds. The other therapeutically active compounds may be for the treatment of the same disease or condition as the compounds of formula (I) or a different disease or condition. The therapeutically active compounds may be administered simultaneously, sequentially or separately.

The compounds of formula (I) may be administered with other active compounds for the treatment of obesity and/or diabetes, for example insulin and insulin analogs, gastric lipase inhibitors, pancreatic lipase inhibitors, sulfonyl ureas and analogs, biguanides, α2 agonists, glitazones, PPAR-γ agonists, mixed PPAR-α/γ agonists, RXR agonists, fatty acid oxidation inhibitors, α-glucosidase inhibitors, dipeptidyl peptidase IV inhibitors, GLP-1 agonists e.g. GLP-1 analogues and mimetics, β-agonists, phosphodiesterase inhibitors, lipid lowering agents, glycogen phosphorylase inhibitors, antiobesity agents e.g. pancreatic lipase inhibitors, MCH-1 antagonists and CB-1 antagonists (or inverse agonists), amylin antagonists, lipoxygenase inhibitors, somostatin analogs, glucokinase activators, glucagon antagonists, insulin signalling agonists, PTP1B inhibitors, gluconeogenesis inhibitors, antilypolitic agents, GSK inhibitors, galanin receptor agonists, anorectic agents, CCK receptor agonists, leptin, serotonergic/dopaminergic antiobesity drugs, reuptake inhibitors e.g. sibutramine, CRF antagonists, CRF binding proteins, thyromimetic compounds, aldose reductase inhibitors, glucocorticoid receptor antagonists, NHE-1 inhibitors or sorbitol dehydrogenase inhibitors.

Combination therapy comprising the administration of a compound of formula (I), or a pharmaceutically acceptable salt thereof, and at least one other antiobesity agent represents a further aspect of the invention.

The present invention also provides a method for the treatment of obesity in a mammal, such as a human, which method comprises administering an effective amount of a compound of formula (I), or a pharmaceutically acceptable salt thereof, and another antiobesity agent, to a mammal in need thereof.

The invention also provides the use of a compound of formula (I), or a pharmaceutically acceptable salt thereof, and another antiobesity agent for the treatment of obesity.

The invention also provides the use of a compound of formula (I), or a pharmaceutically acceptable salt thereof, in the manufacture of a medicament for use in combination with another antiobesity agent, for the treatment of obesity.

The compound of formula (I), or a pharmaceutically acceptable salt thereof, and the other antiobesity agent(s) may be co-administered or administered sequentially or separately.

Co-administration includes administration of a formulation which includes both the compound of formula (I), or a pharmaceutically acceptable salt thereof, and the other antiobesity agent(s), or the simultaneous or separate administration of different formulations of each agent. Where the pharmacological profiles of the compound of formula (I), or a pharmaceutically acceptable salt thereof, and the other antiobesity agent(s) allow it, coadministration of the two agents may be preferred.

The invention also provides the use of a compound of formula (I), or a pharmaceutically acceptable salt thereof, and another antiobesity agent in the manufacture of a medicament for the treatment of obesity.

The invention also provides a pharmaceutical composition comprising a compound of formula (I), or a pharmaceutically acceptable salt thereof, and another antiobesity agent, and a pharmaceutically acceptable carrier. The invention also encompasses the use of such compositions in the methods described above.

GPR119 agonists are of particular use in combination with centrally acting antiobesity agents.

The other antiobesity agent for use in the combination therapies according to this aspect of the invention is preferably a CB-1 modulator, e.g. a CB-1 antagonist or inverse agonist. Examples of CB-1 modulators include SR141716 (rimonabant) and SLV-319 ((4S)-(−)-3-(4-chlorophenyl)-N-methyl-N-[(4-chlorophenyl)sulfonyl]-4-phenyl-4,5-dihydro-1H-pyrazole-1-carboxamide); as well as those compounds disclosed in EP576357, EP656354, WO 03/018060, WO 03/020217, WO 03/020314, WO 03/026647, WO 03/026648, WO 03/027076, WO 03/040105, WO 03/051850, WO 03/051851, WO 03/053431, WO 03/063781, WO 03/075660, WO 03/077847, WO 03/078413, WO 03/082190, WO 03/082191, WO 03/082833, WO 03/084930, WO 03/084943, WO 03/086288, WO 03/087037, WO 03/088968, WO 04/012671, WO 04/013120, WO 04/026301, WO 04/029204, WO 04/034968, WO 04/035566, WO 04/037823 WO 04/052864, WO 04/058145, WO 04/058255, WO 04/060870, WO 04/060888, WO 04/069837, WO 04/069837, WO 04/072076, WO 04/072077, WO 04/078261 and WO 04/108728, and the references disclosed therein.

Other diseases or conditions in which GPR119 has been suggested to play a role include those described in WO 00/50562 and U.S. Pat. No. 6,468,756, for example cardiovascular disorders, hypertension, respiratory disorders, gestational abnormalities, gastrointestinal disorders, immune disorders, musculoskeletal disorders, depression, phobias, anxiety, mood disorders and Alzheimer's disease.

All publications, including, but not limited to, patents and patent application cited in this specification, are herein incorporated by reference as if each individual publication were specifically and individually indicated to be incorporated by reference herein as fully set forth.

The invention will now be described by reference to the following examples which are for illustrative purposes and are not to be construed as a limitation of the scope of the present invention.

### EXAMPLES

Abbreviations and acronyms: Boc: tert-Butoxycarbonyl; t-Bu: tert-Butyl; DCE: 1,2-Dichloroethane; DCM: Dichloromethane; DMAP: 4-Dimethylaminopyridine; DMF: N,N-Dimethylformamide; h: hour; DMSO: Dimethylsulfoxide; EDC: 13-Dimethylaminopropyl)-3-ethylcarbodiimide hydrochloride; Et Ethyl; HOBt 1-Hydroxybenzotriazole hydrate; HPLC: High performance liquid chromatography; mCPBA: 3-Chloroperoxybenzoic acid; Me: Methyl; mins: minutes; Ph: Phenyl; RP-HPLC: Reverse phase high performance liquid chromatography; rt: room temperature; TFA: Trifluoroacetic acid; THF: Tetrahydrofuran.

LCMS Method 1

LCMS data were obtained as follows: Waters Xterra MS C18, 5 μm (4.6×50 mm, flow rate 1.5 ml/min) eluting with a H₂O-MeCN gradient containing 0.1% v/v ammonia over 12 minutes with UV detection at 215 and 254 nm. Gradient information: 0.0-8.0 min: Ramp from 95% H₂O-5% MeCN to 5% H₂O-95% MeCN; 8.0-9.9 min: Hold at 5% H₂O-95% MeCN; 9.9-10.0 min: Return to 95% H₂O-5% MeCN; 10.0-12.0 min: Hold at 95% H₂O-5% MECN. Mass spectra were obtained using an electrospray ionization source in either the positive (ESI) or negative (ESI) mode.

LCMS Method 2

LCMS data were obtained as follows: Waters Atlantis C18, 3μ (3.0×20 mm, flow rate 0.85 ml/min) eluting with a H₂O-MeCN gradient containing 0.1% v/v HCO₂H over 6.5 min with UV detection at 220 nm. Gradient information: 0.0-0.3 min 100% H₂O; 0.3-4.25 min: Ramp to 10% H₂O-90% CH₃CN; 4.25 min-4.4 min: Ramp to 100% CH₃CN; 4.4-4.9 min: Hold at 100% MECN; 4.9-5.0 min: Return to 100% H₂O; 5.00-6.50 min: Hold at 100% H₂O. The mass spectra were obtained using an electrospray ionisation source in either the positive (ESI⁺) ion or negative ion (ESI⁻) mode.

### Preparation 1: 4-Carbamoylmethoxypiperidine-1-carboxylic acid tert-butyl ester

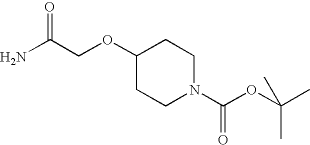

A solution of 4-carboxymethoxypiperidine-1-carboxylic acid tert-butyl ester (14.13 g, 54.7 mmol) and triethylamine (7.68 mL, 65.6 mmol) in anhydrous THF (250 mL) was cooled to 0° C. and isobutylchloroformate (8.511 mL, 65.6 mmol) introduced dropwise. After stirring at 0° C. for 30 min, the reaction mixture was cooled to −20° C. and added rapidly via cannula to a solution of 0.7M ammonia in anhydrous DCM (250 mL, 180 mmol) at −70° C. The reaction was allowed to warm to rt and stirred for 1 h. The mixture was diluted with DCM (250 mL) and washed with saturated aqueous NaHCO₃ (200 mL), 0.5M HCl (200 mL) and brine (200 mL) then dried (MgSO₄). The solvent was evaporated and the residue purified by flash chromatography (IH-THF 3:7) to afford the title compound: δ_{H} (CDCl₃) 1.49 (9H, s), 1.53-1.60 (2H, m), 1.85-1.92 (2H, m), 3.11 (2H, m), 3.58 (1H, m), 3.76-3.83 (2H, m), 3.98 (2H, s), 6.19 (1H, bs), 6.56 (1H, bs).

### Preparation 2: 4-Cyanomethoxypiperidine-1-carboxylic acid tert-butyl ester

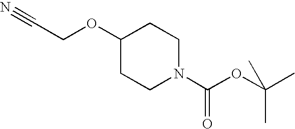

A solution of 4-carbamoylmethoxypiperidine-1-carboxylic acid tert-butyl ester (235 mg, 0.91 mmol) and triethylamine (140 μL, 1 mmol) in anhydrous DCM (5 mL) was cooled to 0° C. and a solution of trichloroacetyl chloride (174 mg, 0.96 mmol) in anhydrous DCM added dropwise. The reaction mixture was stirred at rt for 1 h, the solvent was removed and the residue purified by flash chromatography (1H-EtOAc, 1:1) to afford the title compound: δ_{H}(CDCl₃) 1.50 (9H, s), 1.58-1.65 (2H, m), 1.89-1.95 (2H, m), 3.20 (2H, m), 3.74-3.79 (3H, m), 4.33 (2H, s).

### Preparation 3: 4-(N-Hydroxycarbamimidoylmethoxy)piperidine-1-carboxylic acid tert-butyl ester

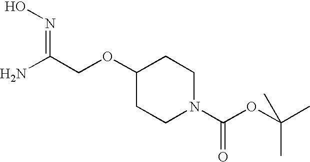

A solution of potassium carbonate (119 mg, 0.86 mmol) and NH₂OH.HCl (119 mg, 1.71 mmol) in water (0.5 mL) was added to 4-cyanomethoxypiperidine-1-carboxylic acid tert-butyl ester (206 mg, 0.857 mmol) in ethanol (2 mL). The mixture was heated at 75° C. for 0.75 h, cooled and the ethanol evaporated. The residue was diluted with EtOAc (50 mL) and washed with water (2×10 mL) and brine (10 mL) then dried (MgSO₄). The solvent was removed to afford the title compound. δ_{H} (CDCl₃) 1.50 (9H, s), 1.50-1.60 (2H, m), 1.85-1.92 (2H, m), 3.13 (2H, m), 3.56 (1H, m), 3.77-3.84 (2H, m), 4.05 (2H, s), 4.82 (2H, bs); MH⁺ 274.0, retention time 2.70 min (Method 2).

### Preparation 4: 5-Methylsulfanylbenzofuran-2-carboxylic acid ethyl ester

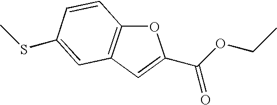

Me₂S₂ (5.58 mL, 62.0 mmol) and t-BuONO (0.59 mL, 5.0 mmol) were added to a stirred solution of 5-aminobenzofuran-2-carboxylic acid ethyl ester (0.85 g, 4.1 mmol) in DCE (60 mL). The mixture was heated to 50° C., then a solution of 5-aminobenzofuran-2-carboxylic acid ethyl ester (7.62 g, 37.2 mmol) in DCE (20 mL) was added concurrently with t-BuONO (5.35 mL, 44.6 mmol), the temperature being maintained at 50° C. After 45 min, the reaction was cooled to rt, before being quenched with H₂O. The organic phase was washed with 1M HCl, before being dried (MgSO₄). Filtration, solvent evaporation, and column chromatography afforded the title compound. δ_{H} (CDCl₃) 1.45 (3H, t), 2.57 (3H, s), 4.47 (2H, q), 7.42 (1H, dd), 7.49 (1H, s), 7.56 (1H, d), 7.61 (1H, d).

### Preparation 5: 5-Amino-benzofuran-2-carboxylic acid ethyl ester

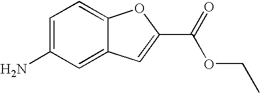

To a solution of 5-nitro-benzofuran-2-carboxylic acid ethyl ester (9.26 g, 39.4 mmol) in EtOAc (350 mL) was added Pd/C (994 mg, 0.93 mmol). The reaction mixture was stirred under an atmosphere of hydrogen for 24 h. The mixture was filtered through celite, washing with EtOAc. The filtrate was concentrated in vacuo to give the title compound: m/z (ES⁺)=206.03 [M+H]⁺.

### Preparation 6: 5-Methylsulfanyl-benzofuran-2-carboxylic acid ethyl ester

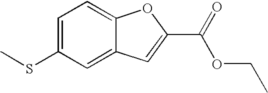

To a solution of 5-amino-benzofuran-2-carboxylic acid ethyl ester (Preparation 5; 0.85 g, 4.13 mmol) in DCE (anhydrous, 60 mL) was added dimethyldisulfide (5.58 mL, 61.98 mmol) and tert-butylnitrite (0.59 mL, 4.96 mmol). The reaction mixture was heated to 50° C. for 30 min then a solution of 5-amino-benzofuran-2-carboxylic acid ethyl ester (7.62 g, 37.2 mmol) in DCE (anhydrous, 20 mL) was added followed by tert-butylnitrite (5.35 mL, 44.63 mmol), whilst maintaining the temperature at 50° C. The reaction mixture was stirred at this temperature for 45 min then cooled to rt and quenched with water. The layers were separated then the organic layer washed with 1M HCl, dried (MgSO₄), filtered and concentrated in vacuo. The residue was chromatographed (EtOAc-1H, 3:97) to afford the title compound: m/z (ES⁺)=237.04 [M+H]⁺.

### Preparation 7: (5-Methylsulfanyl-benzofuran-2-yl)-methanol

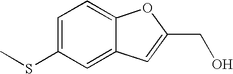

To a solution of 5-methylsulfanyl-benzofuran-2-carboxylic acid ethyl ester (Preparation 6; 100 mg, 0.42 mmol) in EtOH (anhydrous, 2 mL) was added NaBH₄ (48 mg, 1.27 mmol). The reaction mixture was stirred for 24 h at rt then quenched by addition of 1M HCl. The aqueous mixture was extracted with EtOAc then the organic layer washed with brine, dried (MgSO₄), filtered and concentrated in vacuo. The residue was chromatographed (EtOAc-IH, 1:9 then 3:7) furnishing the title compound. δ_{H} (CDCl₃) 1.86 (t, 1H), 2.53 (s, 3H), 4.78 (d, 2H), 6.62 (s, 1H), 7.27 (dd, 1H), 7.39 (d, 1H), 7.51 (d, 1H).

### Preparation 8: 5-Methylsulfanyl-benzofuran-2-carbaldehyde

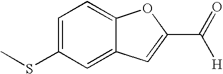

To a solution of (5-methylsulfanylbenzofuran-2-yl)methanol (Preparation 7; 250 mg, 1.29 mmol) in DCM (anhydrous, 13 mL) at 0° C. was added Dess-Martin periodinane (602 mg, 1.42 mmol). The reaction mixture was stirred at rt for 25 min then diluted with Et₂O (35 mL) and poured into 2M NaOH (20 mL). The mixture stirred for 10 min then the layers were separated and the organic layer washed with brine, dried (MgSO₄), filtered and concentrated in vacuo. The residue was chromatographed (EtOAc-IH, 8:92) affording the title compound: m/z (ES⁺)=193.02 [M+H]⁺.

### General Method A

### Synthesis of 4-[5-(1H-indazol-5-yl)[1,2,4]oxadizol-3-ylmethoxy]piperidine-1-carboxylic acid tert-butyl ester (Example 5)

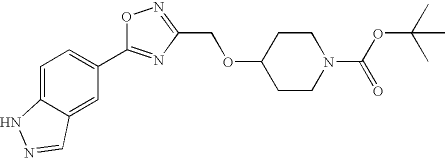

To a solution of 4-(N-hydroxycarbamimidoylmethoxy)piperidine-1-carboxylic acid tert-butyl ester (Preparation 3, 0.36 mmol) in DMSO was added t-BuOK (0.44 mmol). After sonication and heating a workable solution was obtained, and the mixture added to 1H-indazole-5-carboxylic acid methyl ester (0.4 mmol). The reaction mixture was shaken at 60° C. for 15 h. Once the reaction was complete it was neutralised with acetic acid (5 drops) and submitted for HPLC purification. The title compound was obtained as a colourless solid. MH⁺ 400.04, retention time 6.18 min (Method 1).

### General Method B

### Synthesis of 4-(5-benzofuran-2-yl)-[1,2,4]oxadizol-3-ylmethoxy]piperidine-1-carboxylic acid tert-butyl ester (Example 10)

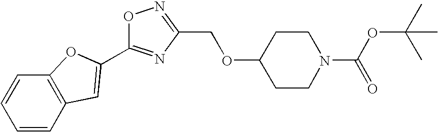

To a solution of benzofuran-2-carboxylic acid (1.2 mmol) in THF (4 mL) was added HOBt (1.2 mmol), followed by EDC (1.2 mmol) and 4-(N-hydroxycarbamimidoylmethoxy)piperidine-1-carboxylic acid tert-butyl ester (Preparation 3, 1.0 mmol). The mixture was stirred at rt overnight. After this time, t-BuOK (5 mmol) was added, and stirring continued for 23 h. The mixture was concentrated and partitioned between EtOAc and water. The organic layer was separated, washed with brine, dried and concentrated. Purification via flash chromatography afforded the title compound as a colourless solid. δ_{H} (400 MHz, CHCl₃) 1.30 (2H, m), 1.50 (9H, s), 1.76 (2H, m), 2.15 (1H, m), 2.75 (2H, m), 2.85 (2H, d), 4.14 (2H, d), 7.39 (1H, t), 7.53 (1H, m), 7.68 (2H, d), 7.77 (1H, d).

### General Method C

### Synthesis of 4-(5-imidazo[1,2-a]pyridine-7-yl-[1,2,4]oxadizol-3-ylmethoxy)piperidine-1-carboxylic acid tert-butyl ester (Example 2)

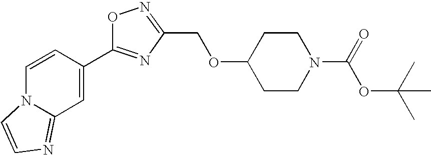

### Step A: Synthesis of 4-[5-(2-aminopyridin-4-yl)[1,2,4]oxadoazol-3-ylmethoxy]piperidine-1-carboxylic acid tert-butyl ester

To a solution of 4-(N-hydroxycarbaimidoylmethoxy)piperidine-1-carboxylic acid tert-butyl ester (Preparation 3, 1.06 g, 3.89 mmol) in THF (30 mL) was added sodium hydride (0.142 g, 3.57 mmol). After effervescence had ceased, 2-aminoisonicotinic acid methyl ester (0.5 g, 3.24 mmol) was added, and the mixture allowed to stir at rt overnight The reaction mixture was diluted with EtOAc and washed with saturated sodium hydrogen carbonate solution, brine, dried and concentrated. Purification via flash chromatography afforded the title compound as a colourless solid. MH⁺ 376.12, retention time 2.61 min (Method 2.)

### Step B: Synthesis of 4-(5-imidazo[1,2-a]pyridine-7-yl-[1,2,4]oxadizol-3-ylmethoxy)piperidine-1-carboxylic acid tert-butyl ester

To a solution of 4-[5-(2-aminopyridin-4-yl)[1,2,4]oxadoazol-3-ylmethoxy]piperidine-1-carboxylic acid tert-butyl ester (0.05 g, 0.13 mmol) in ethanol (2 mL) was added chloro-acetaldehyde (0.069 mL, 0.53 mmol). The reaction mixture was heated to reflux for 17 h. The reaction mixture was cooled, dissolved in ethanol/EtOAc (1:1, 1 mL) and stirred at 30° C. for 3 h. The mixture was concentrated, EtOAc added and the resulting solid filtered. This material was dissolved in saturated sodium hydrogen carbonate solution and extracted with EtOAc. The organic layer was separated, concentrated and the resulting residue purified via flash chromatography. The title compound was obtained as a gum: δ_{H} (400 MHz, CHCl₃) 1.39 (9H, s), 1.57 (2H, m), 1.85 (2H, m), 3.04 (2H, m), 3.65 (1H, m), 3.74 (2H, m), 4.68 (2H, s), 7.46 (1H, d), 7.67 (1H, s), 7.78 (1H, s), 8.21 (1H, d), 8.42 (1H, s).

### General Method D

### Synthesis of 4-[5-(5-nitrobenzofuran-2-yl)-[1,2,4]oxadiazol-3-ylmethyl]piperidine-1-carboxylic acid ter-butyl ester (Example 14)

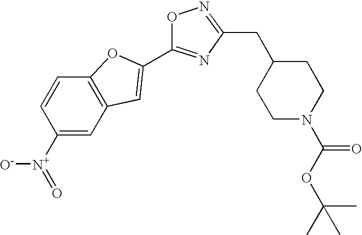

NEt₃ (0.68 mL, 4.83 mmol) was added to a solution of 5-nitrobenzofuran-2-carboxylic acid (1.00 g, 4.83 mmol) in PhMe (30 mL). The stirred mixture was cooled to 0° C., before being treated with i-BuOCOCl (0.63 mL, 4.83 mmol). The reaction was stirred further for 5 min at 0° C., then at ambient temperature for 50 min, before being treated with 4-N-hydroxycarbamimidoylmethyl)piperidine-1-carboxylic acid tert-butyl ester (Preparation 3, 1.03 g, 4.02 mmol) and heated under reflux for 16 h. On cooling to ambient temperature, the reaction was concentrated under reduced pressure and the residue partitioned between EtOAc and saturated aqueous Na₂CO₃. The organic phase was washed with brine, before being dried (MgSO₄). Filtration, solvent evaporation, and column chromatography (1H-EtOAc, 4:1) provided the title compound: MH⁺ 429.16, retention time 4.09 min (Method 2).

### General Method E

### Synthesis of 4-[5-(5-methanesulfinylbenzofuran-2-yl)[1,2,4]oxadiazol-3-ylmethyl]-piperidine-1-carboxylic acid tert-butyl ester (Example 16) and 4-[5-(5-methanesulfonylbenzofuran-2-yl)-[1,2,4]oxadiazol-3-ylmethyl]piperidine-1-carboxylic acid tert-butyl ester (Example 17)

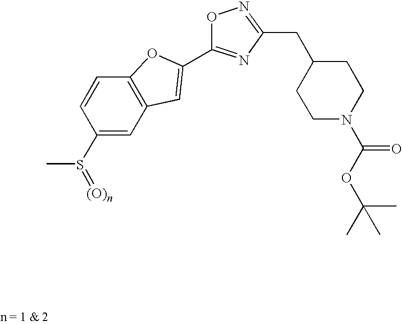

mCPBA (77%, 88 mg, 392 μmol) was added to a stirred solution of 4-[5-(5-methylsulfanylbenzofuran-2-yl)-[1,2,4]oxadiazol-3-ylmethyl]piperidine-1-carboxylic acid tert-butyl ester (Example 15, 112 mg, 261 mol) in CH₂Cl₂. After 1 h, the reaction was quenched with saturated aqueous Na₂CO₃, the organic phase was washed with brine and dried (MgSO₄). Filtration, solvent evaporation and column chromatography (1H-EtOAc, 1:1) yielded the title sulfone. MH⁺ 462.17, retention time 3.77 min (Method 2). Further elution of the column with EtOAc furnished the title sulfoxide: MH⁺ 446.17, retention time 3.59 min (Method 2).

| | | | Prep | | LCMS | LCMS |
|---|---|---|---|---|---|---|
| Ex | Structure | Name | Method | MH+ | RT (min) | Method |
| 1 | 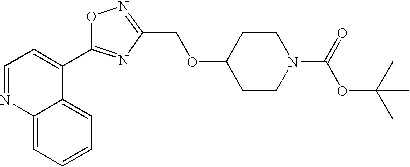 | 4-(5-Quinolin-4-yl- [1,2,4]oxadiazol-3- ylmethoxy)piperidine- 1-carboxylic acid tert- butyl ester | B | 411.09 | 3.92 | 2 |
| 2 | 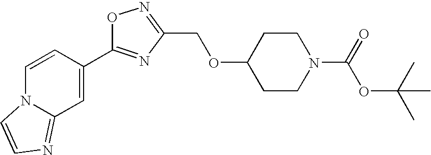 | 4-(5-Imidazo[1,2- a]pyridine-7-yl- [1,2,4]oxadizol-3- ylmethoxy)piperidine- 1-carboxylic acid tert- butyl ester | C | 400.14 | 2.67 | 2 |
| 3 | 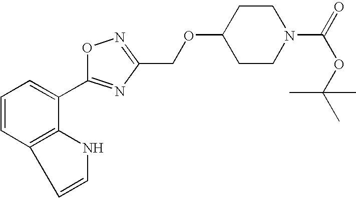 | 4-[5-(1H-Indol-7-yl)- [1,2,4]oxadiazol-3- ylmethoxy]piperidine- 1-carboxylic acid tert- butyl ester | A | 399.07 | 7.75 | 1 |
| 4 | 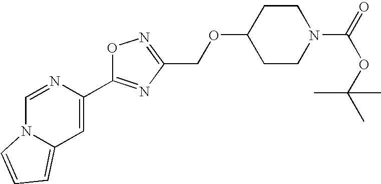 | 4-(5-Pyrrolo[1,2- c]pyrimidin-3-yl- [1,2,4]oxadiazol-3- ylmethoxy)piperidine- 1-carboxylic acid tert- butyl ester | A | 400.04 | 6.43 | 1 |
| 5 | 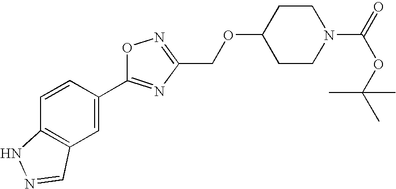 | 4-[5-(1H-Indazol-5- yl)-[1,2,4]oxadizol-3- ylmethoxy]piperidine- 1-carboxylic acid tert- butyl ester | A | 400.04 | 6.18 | 1 |
| 6 | 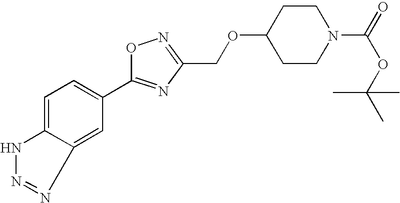 | 4-[5-(1H-Benzotriazol- 5-yl)-[1,2,4]oxadiazol- 3-ylmethoxy]- piperidine-1- carboxylic acid tert- butyl ester | A | 401.06 | 3.87 | 1 |
| 7 | 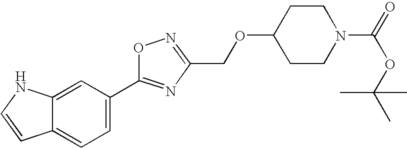 | 4-[5-(1H-Indol-6-yl)- [1,2,4]oxadiazol-3- ylmethoxy]piperidine- 1-carboxylic acid tert- butyl ester | A | 399.07 | 7.10 | 1 |
| 8 | 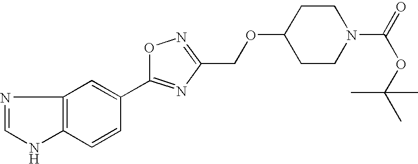 | 4-[5-(1H- Benzoimidazol-5-yl)- [1,2,4]oxadiazol-3- ylmethoxy]piperidine- 1-carboxylic acid tert- butyl ester | A | 400.04 | 5.55 | 1 |
| 9 | 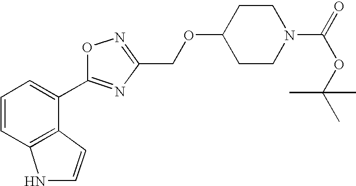 | 4-[5-(1H-Indol-4-yl)- [1,2,4]oxadiazol-3- ylmethoxy]piperidine- 1-carboxylic acid tert- butyl ester | A | 399.07 | 6.99 | 1 |
| 10 | 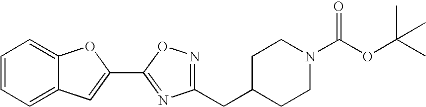 | 4-(5-Benzofuran-2-yl)- [1,2,4]oxadizol-3- ylmethoxy]piperidine- 1-carboxylic acid tert- butyl ester | B | 384.15 | 4.26 | 2 |
| 11 | 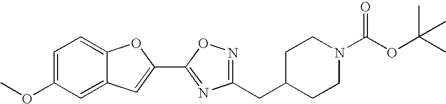 | 4-[5-(5-Methoxy- benzofuran-2-yl)- [1,2,4]oxadizol-3- ylmethoxy]piperidine- 1-carboxylic acid tert- butyl ester | B | 414.11 | 4.32 | 2 |
| 12 | 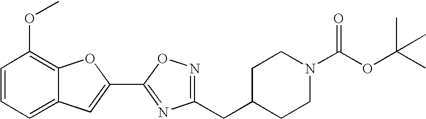 | 4-[5-(7-Methoxy- benzofuran-2-yl)- [1,2,4]oxadizol-3- ylmethoxy]piperidine- 1-carboxylic acid tert- butyl ester | B | 414.12 | 4.20 | 2 |
| 13 | 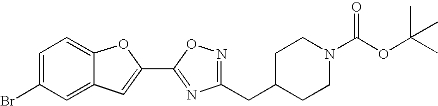 | 4-[5-(5-Bromo- benzofuran-2-yl)- [1,2,4]oxadizol-3- ylmethoxy]piperidine- 1-carboxylic acid tert- butyl ester | B | 462.04 | 4.16 | 2 |
| 14 | 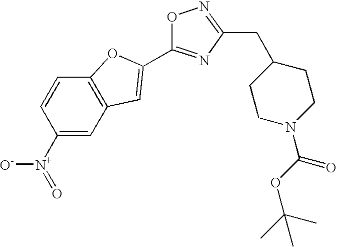 | 4-[5-(5- Nitrobenzofuran-2-yl)- [1,2,4]oxadiazol-3- ylmethoxy]piperidine- 1-carboxylic acid tert- butyl ester | D | 429.16 | 4.09 | 2 |
| 15 | 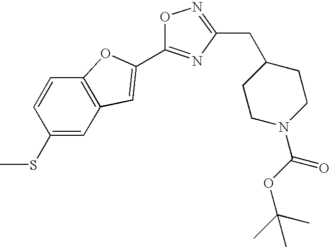 | 4-[5-(5- Methylsulfanyl- benzofuran-2-yl)- [1,2,4]oxadiazol-3- ylmethyl]piperidine-1- carboxylic acid tert- butyl ester | C (Step A) | 430.23 | 4.32 | 2 |
| 16 | 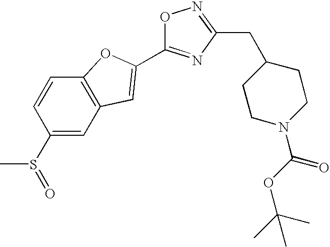 | 4-[5-(5- Methanesulfinyl- benzofuran-2-yl)- [1,2,4]oxadiazol-3- ylmethyl]piperidine-1- carboxylic acid tert- butyl ester | E | 446.17 | 3.59 | 2 |
| 17 | 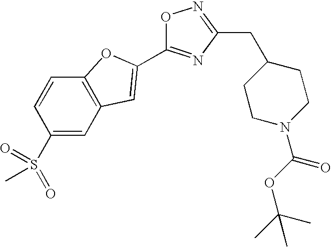 | 4-[5-(5- Methanesulfonyl- benzofuran-2-yl)- [1,2,4]oxadiazol-3- ylmethyl]piperidine-1- carboxylic acid tert- butyl ester | E | 462.17 | 3.77 | 2 |
| 18 | 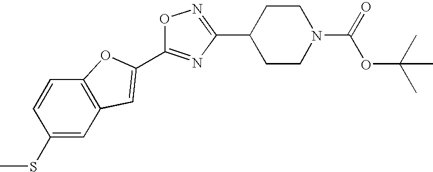 | 4-[5-(5- Methylsulfanyl- benzofuran-2-yl)- [1,2,4]oxadiazol-3-yl]- piperidine-1- carboxylic acid tert- butyl ester | C (Step A) | 416.19 | 4.26 | 2 |
| 19 | 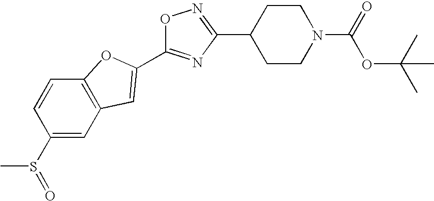 | 4-[5-(5- Methanesulfinyl- benzofuran-2-yl)- [1,2,4]oxadiazol-3-yl]- piperidine-1- carboxylic acid tert- butyl ester | E | 432.16 | 3.56 | 2 |
| 20 | 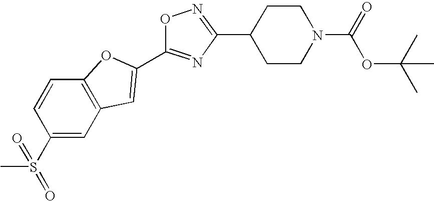 | 4-[5-(5- Methanesulfonyl- benzofuran-2-yl)- [1,2,4]oxadiazol-3-yl]- piperidine-1- carboxylic acid tert- butyl ester | E | 448.19 | 3.72 | 2 |
| 21 | 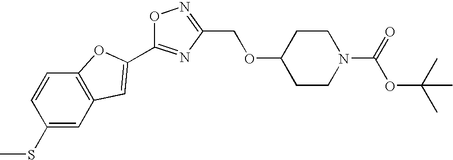 | 4-[5-(5- Methylsulfanyl- benzofuran-2-yl)- [1,2,4]oxadiazol-3- ylmethoxy]piperidine- 1-carboxylic acid tert- butyl ester | C (Step A) | 446.20 | 4.14 | 2 |
| 22 | 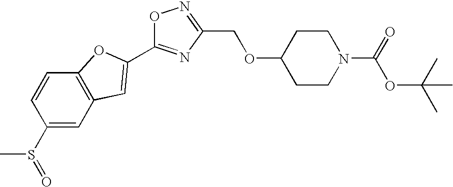 | 4-[5-(5- Methanesulfinyl- benzofuran-2-yl)- [1,2,4]oxadiazol-3- ylmethoxy]piperidine- 1-carboxylic acid tert- butyl ester | E | 462.15 | 3.51 | 2 |
| 23 | 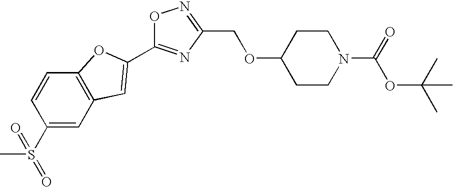 | 4-[5-(5- Methanesulfonyl- benzofuran-2-yl)- [1,2,4]oxadiazol-3- ylmethoxy]piperidine- 1-carboxylic acid tert- butyl ester | E | 478.15 | 3.67 | 2 |
| 24 | 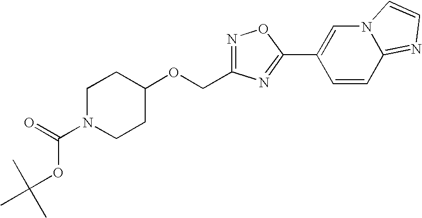 | 4-(5-Imidazo[1,2- a]pyridin-6-yl- [1,2,4]oxadiazol-3- ylmethoxy)piperidine- 1-carboxylic acid tert- butyl ester | B | 399.99 | 5.38 | 1 |
| 25 | 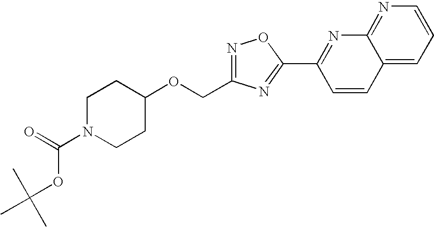 | 4-(5- [1,8]Naphthyridin-2- yl-[1,2,4]oxadiazol-3- ylmethoxy)piperidine- 1-carboxylic acid tert- butyl ester | B | 411.99 | 5.27 | 1 |
| 26 | 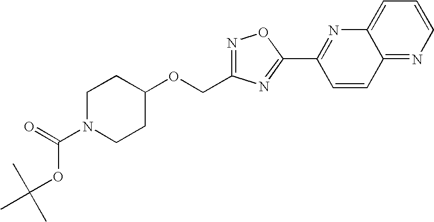 | 4-(5- [1,5]Naphthyridin-2- yl-[1,2,4]oxadiazol-3- ylmethoxy)piperidine- 1-carboxylic acid tert- butyl ester | B | 411.99 | 5.52 | 1 |
| 27 | 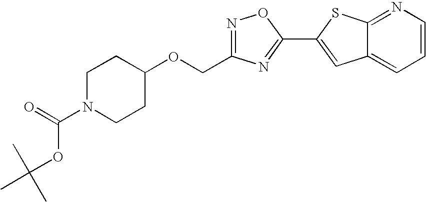 | 4-(5-Thieno[2,3- b]pyridin-2-yl- [1,2,4]oxadiazol-3- ylmethoxy)piperidine- 1-carboxylic acid tert- butyl ester | B | 416.93 | 6.21 | 1 |
| 28 | 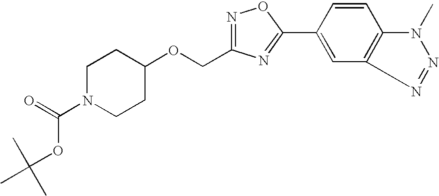 | 4-[5-(1-Methyl-1H- benzotriazol-5-yl)- [1,2,4]oxadiazol-3- ylmethoxy]piperidine- 1-carboxylic acid tert- butyl ester | B | 414.99 | 5.67 | 1 |
| 29 | 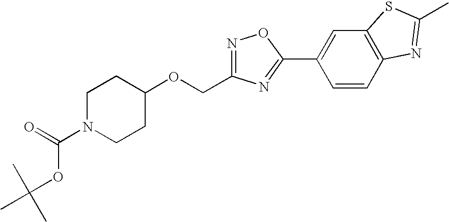 | 4-[5-(2-Amino- benzothiazol-6-yl)- [1,2,4]oxadiazol-3- ylmethoxy]piperidine- 1-carboxylic acid tert- butyl ester | B | 431.96 | 5.60 | 1 |
| 30 | 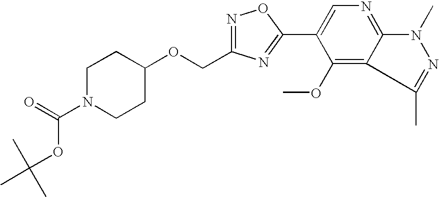 | 4-[5-(4-Methoxy-1,3- dimethyl-1H- pyrazolo[3,4- b]pyridin-5-yl)- [1,2,4]oxadiazol-3- ylmethoxy]piperidine- 1-carboxylic acid tert- butyl ester | B | 459.01 | 6.27 | 1 |
| 31 | 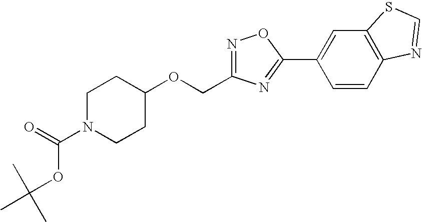 | 4-(5-Benzothiazol-6- yl-[1,2,4]oxadiazol-3- ylmethoxy)piperidine- 1-carboxylic acid tert- butyl ester | B | 416.96 | 6.42 | 1 |

### Example 32

### 4-[2-(5-Methylsulfanylbenzofuran-2-yl)vinyl]piperidin-1-carboxylic acid tert-butyl ester

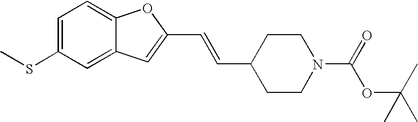

To a cooled (−5° C.) suspension of [[1-[(1,1-dimethylethoxy)carbonyl]-4-piperidinyl]methyl]triphenylphosphonium iodide (744 mg, 1.27 mmol) in THF (anhydrous, 18 mL) was added LHMDS (1.27 mL, 1.27 mmol) dropwise then the resulting solution stirred at −5° C. for 2 h. 5-Methylsulfanyl-benzofuran-2-carbaldehyde (Preparation 8; 221 mg, 1.15 mmol) was added in one portion and the reaction mixture allowed to warm to rt before stirring was continued for 24 h. The reaction was quenched by addition of water then diluted with EtOAc. The layers were separated then the organic layer washed with brine, dried (MgSO₄), filtered and concentrated in vacuo. The residue was chromatographed (EtOAc-1H, 7:93) to afford the title compound: m/z (ES⁺)=374.20 [M+H]⁺.

### Example 33

### 4-[2-(5-Methanesulfinylbenzofuran-2-yl)vinyl]piperidine-1-carboxylic acid tert-butyl ester

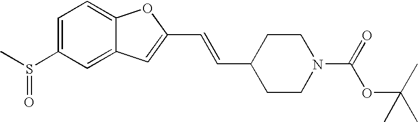

To a solution of 4-[2-(5-methylsulfanylbenzofuran-2-yl)vinyl]piperidine-1-carboxylic acid tert-butyl ester (Example 32; 134 mg, 0.36 mmol) in DCM (anhydrous, 6 mL) was added MCPBA (78 mg, 0.36 mmol). The reaction mixture was stirred for 1 h at rt then saturated aqueous NaHCO₃ was added. The layers were separated and the organic layer washed with saturated aqueous NaHCO₃, brine, dried (MgSO₄), filtered and concentrated in vacuo. The product was chromatographed (EtOAc-1H, 1:1 then EtOAc) to furnish the title compound: m/z (ES⁺)=390.16 [M+H]⁺.

### Example 34

### 4-[2-(5-Methanesulfinylbenzofuran-2-yl)ethyl]piperidine-1-carboxylic acid tert-butyl ester

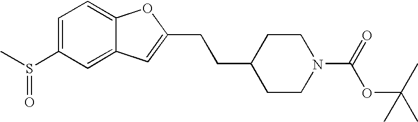

To a solution of 4-[2-(5-methanesulfinylbenzofuran-2-yl)vinyl]piperidine-1-carboxylic acid tert-butyl ester (Example 33; 97 mg, 0.25 mmol) in EtOH (6 mL) was added Pd/C (26 mg, 0.02 mmol) then the reaction mixture stirred under an atmosphere of hydrogen for 24 h. The mixture was filtered through celite, washing with EtOAc and the filtrate concentrated in vacuo. The product was chromatographed (EtOAc-1H, 1:1 then EtOAc) to give the title compound: m/z (ES⁺)=392.19 [M+H]⁺.

### Example 35

### 4-[2-(5-Methanesulfonylbenzofuran-2-yl)ethyl]piperidine-1-carboxylic acid tert-butyl ester

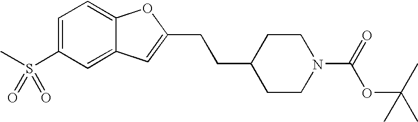

To a solution of 4-[2-(5-methanesulfinylbenzofuran-2-yl)ethyl]piperidine-1-carboxylic acid tert-butyl ester (Example 34; 62 mg, 0.16 mmol) in DCM (anhydrous, 5 mL) was added MCPBA (34 mg, 0.16 mmol) then the reaction mixture stirred for 45 min. Saturated aqueous Na₂CO₃ was added then the layers were separated. The organic layer was washed with brine, dried (MgSO₄), filtered and concentrated in vacuo to give the title compound which needed no further purification: m/z (ES⁺)=408.20 [M+H]⁺.

### Example 36

### 4-(5-Methylsulfanylbenzofuran-2-ylmethoxymethyl)piperidine-1-carboxylic acid tert-butyl ester

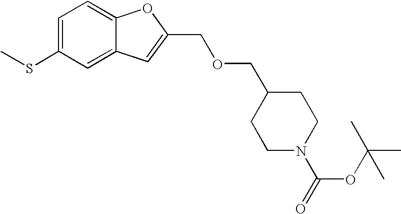

To a solution of (5-methylsulfanylbenzofuran-2-yl)methanol (Preparation 7; 130 mg, 0.67 mmol) in THF (anhydrous, 5 mL) was added NaH (24 mg, 1.01 mmol) then the reaction mixture stirred for 10 min. 4-Methanesulfonyloxymethylpiperidine-1-carboxylic acid tert-butyl ester (394 mg, 1.43 mmol) was added followed by Bu₄N⁺I⁻ (25 mg, 0.07 mmol). The reaction mixture was heated at 90° C. in the microwave for 1 h then more NaH (24 mg, 1.01 mmol) and 4-methanesulfonyloxymethyl-piperidine-1-carboxylic acid tert-butyl ester (197 mg, 0.67 mmol) were added. The reaction mixture was heated at 90° C. in the microwave for 75 min then the volatiles removed in vacuo. The residue was dissolved in EtOAc then washed with water, brine, dried (MgSO₄), filtered and concentrated under reduced pressure. The product was chromatographed (EtOAc-1H, 13:87) to give the title compound: m/z (ES⁺)=392.16 [M+H]⁺.

### Example 37

### 4-(5-Methanesulfinylbenzofuran-2-ylmethoxymethyl)piperidine-1-carboxylic acid tert-butyl ester

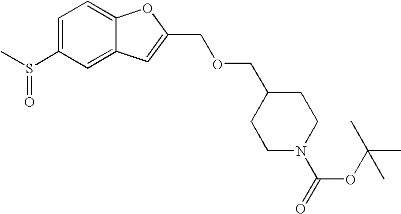

To a solution of 4-(5-methylsulfanylbenzofuran-2-ylmethoxymethyl)piperidine-1-carboxylic acid tert-butyl ester (Example 36; 71 mg, 0.18 mmol) in DCM (anhydrous, 4 mL) was added MCPBA (47 mg, 0.27 mmol). The reaction mixture was stirred for 1 h at rt then saturated aqueous NaHCO₃ added. The layers were separated then the organic layer washed with brine, dried (MgSO₄), filtered and concentrated in vacuo. The product was chromatographed (EtOAc-IH, 1:1 then EtOAc) to give the title compound: m/z (ES⁺)=408.12 [M+H]⁺.

### Example 38

### 4-(5-Methanesulfonylbenzofuran-2-ylmethoxymethyl)piperidine-1-carboxylic acid tert-butyl ester

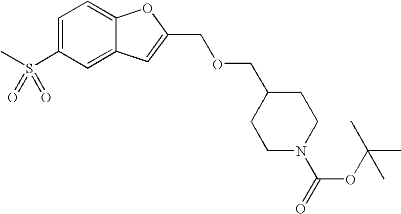

The title compound was obtained as an additional product from Example 37: m/z (ES⁺)=324.08 [M+H —C₅H₈O₂]⁺.

### Example 39

### 4-[2-(5-Methylsulfanylbenzofuran-2-ylmethoxy)ethyl]piperidine-1-carboxylic acid tert-butyl ester

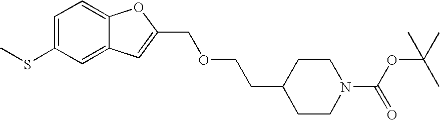

The title compound was prepared from (5-methylsulfanylbenzofuran-2-yl)methanol (Preparation 7) and 4-(2-methanesulfonyloxyethyl)piperidine-1-carboxylic acid tert-butylester using the same procedure and purification as in Example 36: m/z (ES⁺)=406.19 [M+H]⁺.

### Example 40

### 4-[2-(5-Methanesulfinylbenzofuran-2-ylmethoxy)ethyl]piperidine-1-carboxylic acid tert-butyl ester

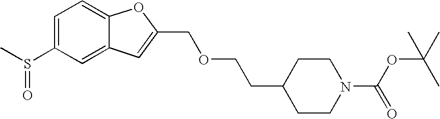

The title compound was prepared from 4-[2-(5-methylsulfanylbenzofuran-2-ylmethoxy)ethyl]piperidine-1-carboxylic acid tert-butyl ester (Example 39) using the same procedure and purification as in Example 37: m/z (ES⁺)=422.19 [M+H]⁺.

### Example 41

### 4-[2-(5-Methanesulfonylbenzofuran-2-ylmethoxy)ethyl]piperidine-1-carboxylic acid tert-butyl ester

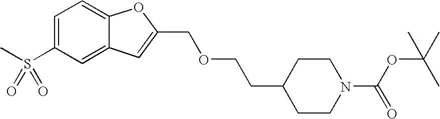

The title compound was obtained as an additional product from Example 40: m/z (ES⁺)=338.13 [M+H —C₅H₈O₂]⁺.

### Example 42

### 4-(5-Methylsulfanylbenzofuran-2-ylmethoxy)piperidine-1-carboxylic acid tert-butyl ester

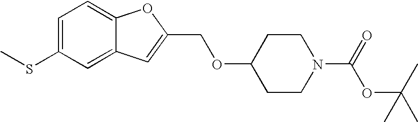

To a solution of (5-methylsulfanylbenzofuran-2-yl)methanol (Preparation 7; 154 mg, 0.79 mmol) in DCM (anhydrous, 6 mL) was added CBr₄ (592 mg, 1.79 mmol). The solution was cooled to 0° C. then PPh₃ (469 mg, 1.79 mmol) was added and the reaction mixture allowed to warm to rt. After stirring for 30 min the reaction mixture was washed with saturated aqueous NaHCO₃ then brine and dried (MgSO₄), filtered and concentrated in vacuo. The crude product was adsorbed onto silica gel then chromatographed (EtOAc-1H, 2:98) to give 2-bromomethyl-5-methylsulfanylbenzofuran as part of a mixture which was used directly without further purification. To a solution of 4-hydroxypiperidine-1-carboxylic acid tert-butyl ester (129 mg, 0.64 mmol) in THF (anhydrous, 4 mL) was added NaH (15 mg, 0.64 mmol) then the reaction mixture stirred at rt for 10 min. 2-Bromomethyl-5-methylsulfanylbenzofuran (82 mg, 0.32 mmol) was added as a solution in THF (anhydrous, 1 mL) followed by Bu₄N⁺I⁻ (24 mg, 0.06 mmol). The mixture was heated at 90° C. in the microwave for 90 min then the volatiles removed in vacuo. The residue was dissolved in EtOAc then washed with water, brine, dried (MgSO₄), filtered and concentrated under reduced pressure. The product was chromatographed (EtOAc-1H, 1:9) to give the title compound: m/z (ES⁺)=378.14 [M+H]⁺.

### Example 43

### 4-(5-Methanesulfinylbenzofuran-2-ylmethoxy)piperidine-1-carboxylic acid tert-butyl ester

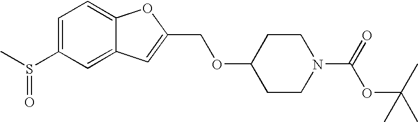

The title compound was prepared from 4-(5-methylsulfanylbenzofuran-2-ylmethoxy)-piperidine-1-carboxylic acid tert-butyl ester (Example 42) using the same procedure and purification as in Example 37: m/z (ES⁺)=338.09 [M+H —C₄H₈]⁺.

### Example 44

### 4-(5-Methanesulfonylbenzofuran-2-ylmethoxy)piperidine-1-carboxylic acid tert-butyl ester

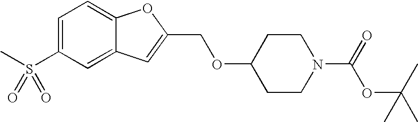

The title compound was obtained as an additional product from Example 43: m/z (ES⁺)=310.11 [M+H —C₅H₈O₂]⁺.

The biological activity of the compounds of the invention may be tested in the following assay systems:

### Yeast Reporter Assay

The yeast cell-based reporter assays have previously been described in the literature (e.g. see Miret J. J. et al, 2002, J. Biol. Chem., 277:6881-6887; Campbell R. M. et al, 1999, Bioorg. Med. Chem. Lett., 9:2413-2418; King K. et al, 1990, Science, 250:121-123); WO 99/14344; WO 00/12704; and U.S. Pat. No. 6,100,042). Briefly, yeast cells have been engineered such that the endogenous yeast G-alpha (GPA1) has been deleted and replaced with G-protein chimeras constructed using multiple techniques. Additionally, the endogenous yeast alpha-cell GPCR, Step 3 has been deleted to allow for a homologous expression of a mammalian GPCR of choice. In the yeast, elements of the pheromone signaling transduction pathway, which are conserved in eukaryotic cells (for example, the mitogen-activated protein kinase pathway), drive the expression of Fus1. By placing β-galactosidase (LacZ) under the control of the Fus1 promoter (Fus1p), a system has been developed whereby receptor activation leads to an enzymatic read-out.

Yeast cells were transformed by an adaptation of the lithium acetate method described by Agatep et al, (Agatep, R. et al, 1998, Transformation of Saccharomyces cerevisiae by the lithium acetate/single-stranded carrier DNA/polyethylene glycol (LiAc/ss-DNA/PEG) protocol. Technical Tips Online, Trends Journals, Elsevier). Briefly, yeast cells were grown overnight on yeast tryptone plates (YT). Carrier single-stranded DNA (10 g), 21 g of each of two Fus1p-LacZ reporter plasmids (one with URA selection marker and one with TRP), 2 μg of GPR119 (human or mouse receptor) in yeast expression vector (2 μg origin of replication) and a lithium acetate/polyethylene glycol/TE buffer was pipetted into an Eppendorf tube. The yeast expression plasmid containing the receptor/no receptor control has a LEU marker. Yeast cells were inoculated into this mixture and the reaction proceeds at 30° C. for 60 min. The yeast cells were then heat-shocked at 42° C. for 15 nm in. The cells were then washed and spread on selection plates. The selection plates are synthetic defined yeast media minus LEU, URA and TRP (SD-LUT). After incubating at 30° C. for 2-3 days, colonies that grow on the selection plates were then tested in the LacZ assay.

In order to perform fluorimetric enzyme assays for β-galactosidase, yeast cells carrying the human or mouse GPR119 receptor were grown overnight in liquid SD-LUT medium to an unsaturated concentration (i.e. the cells were still dividing and had not yet reached stationary phase). They were diluted in fresh medium to an optimal assay concentration and 90 μl of yeast cells are added to 96-well black polystyrene plates (Costar). Compounds, dissolved in DMSO and diluted in a 10% DMSO solution to 10× concentration, were added to the plates and the plates placed at 30° C. for 4 h. After 4 h, the substrate for the β-galactosidase was added to each well. In these experiments, Fluorescein di(β-D-galactopyranoside) was used (FDG), a substrate for the enzyme that releases fluorescein, allowing a fluorimetric read-out. 20 μl per well of 500 μM FDG/2.5% Triton ×100 was added (the detergent was necessary to render the cells permeable). After incubation of the cells with the substrate for 60 min, 20 μl per well of 1M sodium carbonate was added to terminate the reaction and enhance the fluorescent signal. The plates were then read in a fluorimeter at 485/535 nm.

The compounds of the invention give an increase in fluorescent signal of at least ˜1.5-fold that of the background signal (i.e. the signal obtained in the presence of 1% DMSO without compound). Compounds of the invention which give an increase of at least 5-fold may be preferred

cAMP Assay

A stable cell line expressing recombinant human GPR119 was established and this cell line was used to investigate the effect of compounds of the invention on intracellular levels of cyclic AMP (cAMP). The cells monolayers were washed with phosphate buffered saline and stimulated at 37° C. for 30 min with various concentrations of compound in stimulation buffer plus 1% DMSO. Cells were then lysed and cAMP content determined using the Perkin Elmer AlphaScreen™ (Amplified Luminescent Proximity Homogeneous Assay) cAMP kit. Buffers and assay conditions were as described in the manufacturer's protocol. Compounds of the invention showed a concentration-dependant increase in intracellular cAMP level.

Compounds of the invention showed a concentration-dependant increase in intracellular cAMP level and generally had an EC₅₀ of <10 uM. Compounds showing an EC₅₀ of less than 1 uM in the cAMP assay may be preferred.

### In Vivo Feeding Study

The effect of compounds of the invention on body weight and food and water intake may be examined in freely-feeding male Sprague-Dawley rats maintained on reverse-phase lighting. Test compounds and reference compounds are dosed by appropriate routes of administration (e.g. intraperitoneally or orally) and measurements made over the following 24 h. Rats are individually housed in polypropylene cages with metal grid floors at a temperature of 21±4° C. and 55±20% humidity. Polypropylene trays with cage pads are placed beneath each cage to detect any food spillage. Animals are maintained on a reverse phase light-dark cycle (lights off for 8 h from 09.30-17.30 h) during which time the room was illuminated by red light. Animals have free access to a standard powdered rat diet and tap water during a two week acclimatization period. The diet is contained in glass feeding jars with aluminum lids. Each lid has a 3-4 cm hole in it to allow access to the food. Animals, feeding jars and water bottles are weighed (to the nearest 0.1 g) at the onset of the dark period. The feeding jars and water bottles are subsequently measured 1, 2, 4, 6 and 24 h after animals are dosed with a compound of the invention and any significant differences between the treatment groups at baseline compared to vehicle-treated controls.

Selected compounds of the invention showed a statistically significant hypophagic effect at one or more time points at a dose of ≦100 mg/kg.

### Anti-Diabetic Effects of Compounds of the Invention in an In-Vitro Model of Pancreatic Beta Cells (HIT-T15)

### Cell Culture

HIT-T15 cells (passage 60) were obtained from ATCC, and were cultured in RPMI1640 medium supplemented with 10% fetal calf serum and 30 nM sodium selenite. All experiments were done with cells at less than passage 70, in accordance with the literature, which describes altered properties of this cell line at passage numbers above 81 (Zhang H J, Walseth T F, Robertson R P. Insulin secretion and cAMP metabolism in HIT cells. Reciprocal and serial passage-dependent relationships. Diabetes. 1989 January; 38(1):44-8).

cAMP assay

HIT-T15 cells were plated in standard culture medium in 96-well plates at 100,000 cells/0.1 ml/well and cultured for 24 hr and the medium was then discarded. Cells were incubated for 15 min at room temperature with 10 μl stimulation buffer (Hanks buffered salt solution, 5 mM HEPES, 0.5 mM IBMX, 0.1% BSA, pH 7.4). This was discarded and replaced with compound dilutions over the range 0.001, 0.003, 0.01, 0.03, 0.1, 0.3, 1, 3, 10, 30 μM in stimulation buffer in the presence of 0.5% DMSO. Cells were incubated at room temperature for 30 min. Then 75 ul lysis buffer (5 mM HEPES, 0.3% Tween-20, 0.1% BSA, pH 7.4) was added per well and the plate was shaken at 900 rpm for 20 min. Particulate matter was removed by centrifugation at 3000 rpm for 5 min, then the samples were transferred in duplicate to 384-well plates, and processed following the Perldn Elmer AlphaScreen cAMP assay kit instructions. Briefly 25 μl reactions were set up containing 8 μl sample, 5 μl acceptor bead mix and 12 μl detection mix, such that the concentration of the final reaction components is the same as stated in the kit instructions. Reactions were incubated at room temperature for 150 min, and the plate was read using a Packard Fusion instrument. Measurements for cAMP were compared to a standard curve of known cAMP amounts (0.01, 0.03, 0.1, 0.3, 1, 3, 10, 30, 100, 300, 1000 nm) to convert the readings to absolute CAMP amounts. Data was analysed using XLfit 3 software.

Representative compounds of the invention were found to increase cAMP at an EC₅₀ of less than 10 μM. Compounds showing an EC₅₀ of less than 1 μM in the CAMP assay may be preferred.

### Insulin Secretion Assay

HIT-T15 cells were plated in standard culture medium in 12-well plates at 106 cells/1 mV well and cultured for 3 days and the medium was then discarded. Cells were washed x 2 with supplemented Krebs-Ringer buffer (KRB) containing 119 mM NaCl, 4.74 mM KCl, 2.54 mM CaCl₂, 1.19 mM MgSO₄, 1.19 mM KH2PO4, 25 mM NaHCO₃, 10 mM HEPES at pH 7.4 and 0.1% bovine serum albumin. Cells were incubated with 1 ml KRB at 37° C. for 30 min which was then discarded. This was followed by a second incubation with KRB for 30 min, which was collected and used to measure basal insulin secretion levels for each well. Compound dilutions (0.0.1, 0.3, 1, 3, 10 uM) were then added to duplicate wells in 11 ml KRB, supplemented with 5.6 mM glucose. After 30 min incubation at 37° C. samples were removed for determination of insulin levels. Measurement of insulin was done using the Mercodia Rat insulin ELISA kit, following the manufacturers instructions, with a standard curve of known insulin concentrations. For each well insulin levels were subtracted by the basal secretion level from the pro-incubation in the absence of glucose. Data was analysed using XLfit 3 software.

Representative compounds of the invention were found to increase insulin secretion at an EC₅₀ of less than 10 μM. Compounds showing an EC₅₀ of less than 1 μM in the insulin secretion assay may be preferred.

### Oral Glucose Tolerance Tests

The effects of compounds of the invention on oral glucose (Glc) tolerance may be evaluated in male C57BV/6 or male oblob mice. Food is withdrawn 5 h before administration of Glc and remains withdrawn throughout the study. Mice have free access to water during the study. A cut is made to the animals' tails, then blood (20 μL) is removed for measurement of basal Glc levels 45 min before administration of the Glc load. The mice are weighed and dosed orally with test compound or vehicle (20% aqueous hydroxypropyl-β-cyclodextrin or 25% aqueous Gelucire 44/14) 30 min before the removal of an additional blood sample (20 μL) and treatment with the Glc load (2-5 g kg⁻¹ p.o.). Blood samples (20 μL) are taken 25, 50, 80, 120, and 180 min after Glc administration. The 20 μL blood samples for measurement of Glc levels are taken from the cut tip of the tail into disposable micro-pipettes (Dade Diagnostics Inc., Puerto Rico) and the sample added to 480 μL of haemolysis reagent. Duplicate 20 μL aliquots of the diluted haemolysed blood are added to 180 μL of Trinders glucose reagent (Sigma enzymatic (Trinder) colormetric method) in a 96-well assay plate. After mixing, the samples are left at rt for 30 min before being read against Glc standards (Sigma glucose/urea nitrogen combined standard set).

What is claimed is:

1. A compound of formula (I): 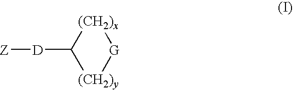
or a pharmaceutically acceptable salt or N-oxide thereof, wherein:
Z represents a group: 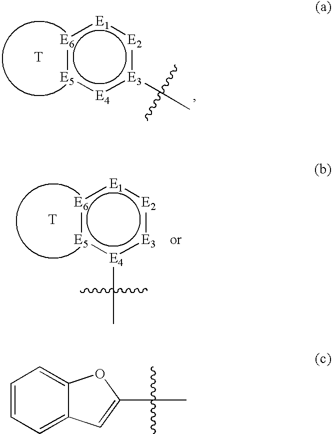
wherein E₁ to E₆ may independently represent either C/CH or N; T represents a five or six membered aryl or nitrogen containing heteroaryl ring, with the proviso that when Z represents the group (a), T does not represent: 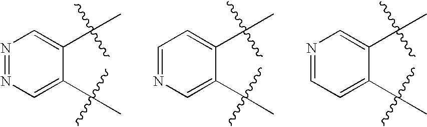
the group Z may optionally be substituted by one or more groups selected from halogen, CF₃, C₁₋₄alkoxy, NR⁴R⁴⁴, S(O)ₘR⁴, SO₂NR⁴R⁴⁴, CONR⁴R⁴⁴, NR¹⁰CONR⁴R⁴⁴, NR¹⁰COR⁴, NR¹⁰SO₂R⁴, nitro, cyano, or a 5- or 6-membered heteroaryl ring; or C₁₋₄alkyl, C₂₋₄alkenyl, or C₂₋₄alkynyl, which may be optionally substituted by hydroxy, NR⁴R⁴⁴, oxo or C₁₋₄alkoxy;
m is 0, 1 or 2;
D represents a group -B-Q-A-, wherein:
Q is a 5- or 6-membered heteroaromatic ring;
A is (CH₂)ₙ, where one CH₂ group may be replaced by O, S, C(O), CH(OH)CH(halo) CH(NR²R³), S(O), S(O)₂ or NR³; two CH₂ groups may be replaced by CH═CH, C(O)O, C(O)S, SC(O), C(O)NR² or OC(O); or three CH₂ groups may be replaced by C(O)CH₂S, C(O)CH₂C(OH) or C(O)CH₂C(O);
n is 0, 1, 2, 3, 4, 5, or 6;
B is a bond, —CH₂═CH₂— or (CH₂)ⱼ;
j is 1, 2 or 3;
or D represents -M-U—V—, wherein:
M and V are independently a bond, an unbranched or a branched C₁₋₃ alkylene or an unbranched or a branched C₂₋₃ alkenylene;
U is selected from CH₂, O, S, CH(OH), CH(halo), CH═CH, C(O), C(O)O, C(O)S, SC(O), C(O)CH₂S, C(O)CH₂C(OH), C(O)CH₂C(O), OC(O), NR², CH(NR²R²²), C(O)NR², S(O) and S(O)₂;
G is CHR⁸ or NR¹;
R¹ is C(O)OR⁵, C(O)NR⁵R¹⁰, C(O)NR⁵R⁵⁵, C₁₋₄alkylene-C(O)OR⁵, C(O)C(O)OR⁵, S(O)₂R⁵, C(O)R⁵ or P(O)(O-Ph)₂; or heterocyclyl or heteroaryl, either of which may optionally be substituted by one or two groups selected from C₁₋₄alkyl, C₁₋₄alkoxy or halogen;
R², R²² and R³ are independently hydrogen or C₁₋₄alkyl;
R⁴ and R⁴ are independently hydrogen, C₁₋₄alkyl, C₃₋₇cycloalkyl, or aryl, which may optionally be substituted with 1 or 2 substituents selected from halo, C₁₋₄alkyl, CF₃, hydroxy, C₁₋₄alkoxy, cyano, and S(O)₂Me; or, taken together, R⁴ and R⁴⁴ may form a 5- or 6-membered heterocyclic ring;
R⁵ and R⁵⁵ are independently C₁₋₈alkyl, C₂₋₈alkenyl or C₂₋₈alkynyl, any of which may be optionally substituted by one or more halo atoms, NR⁶R⁶⁶, OR⁶, C(O)OR⁶, OC(O)R⁶ or cyano, and may contain a CH₂ group that is replaced by O or S; or a C₃₋₇cycloalkyl, aryl, heterocyclyl, heteroaryl, C₁₋₄alkyleneC₃₋₇cycloalkyl, C₁₋₄alkylenearyl, C₁₋₄alkyleneheterocyclyl or C₁₋₄ alkyleneheteroaryl, any of which may be substituted with one or more substituents selected from halo, C₁₋₄alkyl, C₁₋₄fluoroalkyl, OR⁷, CN, NR⁷R⁷⁷, SO₂Me, NO₂ or C(O)OR⁷;
R⁶, R⁶⁶, R⁷, and R⁷⁷ each independently are hydrogen or C₁₋₄alkyl; or, taken together, R⁶ and R⁶⁶ or R⁷ and R⁷⁷ may independently form a 5- or 6-membered heterocyclic ring;
R⁸ is C₃₋₆alkyl;
R¹⁰ is hydrogen or C₁₋₄alkyl;
x is 0, 1, 2 or 3; and
y is 1, 2, 3, 4 or 5; provided that x+y is 2, 3, 4 or 5.

2. A compound according to claim 1, or a pharmaceutically acceptable salt or N-oxide thereof, wherein D represents -B-Q-A-.

3. A compound according to claim 2, or a pharmaceutically acceptable salt or N-oxide thereof, wherein when Q is oxadiazolyl.

4. A compound according to claim 3, or a pharmaceutically acceptable salt or N-oxide thereof, wherein when Q is [1,2,4]oxadiazolyl.

5. A compound according to claim 1, or a pharmaceutically acceptable salt or N-oxide thereof, wherein n is 0, 1 or 2.

6. A compound according to claim 5, or a pharmaceutically acceptable salt or N-oxide thereof, wherein n is 1 or 2.

7. A compound according to claim 6, or a pharmaceutically acceptable salt or N-oxide thereof, wherein A is CH₂, CH₂O or CH₂NR³.

8. A compound according to claim 1, or a pharmaceutically acceptable salt or N-oxide thereof, wherein B represents a bond.

9. A compound according to claim 1, or a pharmaceutically acceptable salt or N-oxide thereof, wherein D represents -M-U—V—.

10. A compound according to claim 9, or a pharmaceutically acceptable salt or N-oxide thereof, wherein U represents CH₂, O or NR².

11. A compound according to claim 9, or a pharmaceutically acceptable salt or N-oxide thereof, wherein M and V are independently C₁₋₃ alkylene.

12. A compound according to claim 1, or a pharmaceutically acceptable salt or N-oxide thereof, wherein R¹ is C(O)OR⁵, C(O)NR⁵R¹⁰ or heteroaryl.

13. A compound according to claim 12, or a pharmaceutically acceptable salt or N-oxide thereof, wherein R¹ is C(O)OR⁵.

14. A compound according to claim 1, or a pharmaceutically acceptable salt or N-oxide thereof, wherein Z represents the group (a).

15. A compound according to claim 1, or a pharmaceutically acceptable salt or N-oxide thereof, wherein Z represents the group (b).

16. A compound according to claim 1, or a pharmaceutically acceptable salt or N-oxide thereof, wherein Z represents the group (c).

17. A compound of formula (I) as defined in any one of Examples 1 to 44, or a pharmaceutically acceptable salt or N-oxide thereof.

18. A pharmaceutical composition comprising a compound according to claim 1, or a pharmaceutically acceptable salt or N-oxide thereof and a pharmaceutically acceptable carrier.

19. A method for the treatment of a disease or condition in which GPR119 plays a role comprising a step of administering to a subject in need thereof an effective amount of a compound according to claim 1, or a pharmaceutically acceptable salt or N-oxide thereof.

20. A method for the regulation of satiety comprising a step of administering to a subject in need thereof an effective amount of a compound according to claim 1, or a pharmaceutically acceptable salt or N-oxide thereof.

21. A method for the treatment of obesity comprising a step of administering to a subject in need thereof an effective amount of a compound according to claim 1, or a pharmaceutically acceptable salt or N-oxide thereof.

22. A method for the treatment of diabetes comprising a step of administering to a subject in need thereof an effective amount of a compound according to claim 1, or a pharmaceutically acceptable salt or N-oxide thereof.

23. A method for the treatment of metabolic syndrome (syndrome X), impaired glucose tolerance, hyperlipidemia, hypertriglyceridemia, hypercholesterolemia, low HDL levels or hypertension comprising a step of administering to a patient in need thereof an effective amount of a compound according to claim 1, or a pharmaceutically acceptable salt or N-oxide thereof.

24. (canceled)

25. (canceled)

26. (canceled)

27. A compound of formula (XVI): 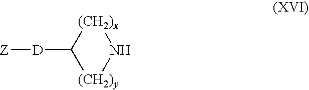
or a salt or protected derivative thereof, wherein Z, D, x and y are as defined in claim 1, with the proviso that when Z represents the group (c), x represents 2 and y represents 2, -M-U—V— does not represent —CH₂—NH—CH₂— or —C(O)—NH—.
